(19) United States
(12) Patent Application Publication
(10) Pub. No.: US 2024/0139280 A1
(43) Pub. Date: May 2, 2024

(54) USE OF HIF-1-ALPHA INHIBITORS IN CANCER IMMUNOTHERAPY

(71) Applicants: ONCOC4, INC., Rockville, MD (US); University of Maryland, Baltimore, Baltimore, MD (US)

(72) Inventors: Yin Wang, Washington, D.C., DC (US); Yang Liu, Potomac, MD (US); Pan Zheng, Potomac, MD (US); Martin Devenport, Gaithersburg, MD (US); Yan Liu, Washington, DC (US); Christopher Bailey, Merritt Island, FL (US)

(21) Appl. No.: 18/286,334

(22) PCT Filed: Apr. 13, 2022
(86) PCT No.: PCT/US2022/024659

§ 371 Date: Oct. 10, 2023

Related U.S. Application Data
(60) Provisional application No. 63/174,848, filed on Apr. 14, 2021.

(51) Int. Cl. A61K 38/12; A61K 39/395; A61P 35/00

(52) U.S. Cl. CPC A61K 38/12; A61K 39/3955; A61P 35/00; A61K 2039/505

(57) ABSTRACT

The present invention relates to the use of hypoxia-inducible factor (HIF) inhibitors in cancer immunotherapy. Specifically, the disclosure provides methods of treating a cancer in a subject in need of cancer immunotherapy, comprising administering a HIF-1a inhibitor to the subject and a second cancer immunotherapeutic agent to the subject, wherein the HIF-1a inhibitor comprising echinomycin, and wherein the second cancer immunotherapeutic agent comprising an anti-CTLA-4 antibody including Ipilimumab or Tremelimumab.

### FIELD OF THE INVENTION

The present invention relates to the use of hypoxia-inducible factor-1-α (HIF-1α) inhibitors in cancer immunotherapy.

### BACKGROUND OF THE INVENTION

Current strategies of immunotherapy, articulated as immune checkpoint blockade, aim to release physiological immune tolerance checkpoints, thereby providing the benefit of an immunotherapeutic effect. As such, immune-related adverse events (irAE) are considered the necessary price for immunotherapy. The relative risk/benefit ratio depends on the significance of the immune checkpoint in immune tolerance vs tumor evasion of host immunity. The programmed death-1 (PD-1):programmed death ligand-1 (PD-L1) interaction is less critical than cytotoxic T-lymphocyte-associated protein 4 (CTLA-4) for immune tolerance, as CTLA-4 inactivation leads to more severe autoimmune diseases than that of PD-1 (Nishimura et al., 1999; Nishimura et al., 2001; Walunas et al., 1994; Waterhouse et al., 1995). Correspondingly, monoclonal antibodies (mAbs) targeting PD-1 and PD-L1 are less toxic than those targeting cytotoxic T-lymphocyte antigen 4 (CTLA-4) (Larkin et al., 2019). In terms of therapeutic efficacy, anti-CTLA-4+anti-PD-1 combination therapy is considered the most effective immunotherapy strategy (Larkin et al., 2019). Yet the combination has not been widely adopted, because it substantially increases rates of severe irAEs (Larkin et al., 2019) to 50-90% depending on therapeutic setting (Amaria et al., 2018; Blank et al., 2018; Hellmann et al., 2018; Motzer et al., 2018). Thus, a major challenge for cancer immunotherapy is to eliminate irAE without compromising synergistic cancer immunotherapeutic effects of dual-immune checkpoint blockade.

In the tumor microenvironment (TME), tumor cells and tumor-infiltrated myeloid subsets express PD-L1 in response to environmental cues including cytokines, hypoxia, or growth factors (Anderson et al., 2017; Noman et al., 2014; Zerdes et al., 2018). PD-L1/B7-H1 causes T cell apoptosis (Dong et al., 2002) and/or exhaustion upon binding PD-1 (Barber et al., 2006). Consequently, the PD-1:PD-L1 interaction suppresses T cell-mediated anticancer immunity in the TME, and blocking this interaction reinvigorates immune rejection of tumor cells (Hirano et al., 2005). Though irAEs resulting from anti-PD-1/PD-L1 mAbs are generally less severe than those from anti-CTLA-4 mAbs (Larkin et al., 2015; Wang et al., 2018), PD-1/PD-L1 blockade does lead to significant irAE and administering anti-PD-1 mAbs concurrently with anti-CTLA-4 mAbs substantially worsens irAE incidence and severity (Hodi et al., 2016; Larkin et al., 2019; Morganstein et al., 2017; Naidoo et al., 2017; Postow et al., 2015). Accordingly, there is a need in the art for safer anti-PD-1/PD-L1- and anti-PD-1/PD-L1/anti-CTLA-4-based cancer immunotherapies that target the TME while reducing irAEs.

### SUMMARY OF THE INVENTION

Provided herein is a method of treating a cancer in a subject. The method may comprise administering a HIF-1α inhibitor to the subject. The method may comprise administering a HIF-1α inhibitor and a second cancer immunotherapeutic agent to the subject. Also provided herein is use of a HIF-1α inhibitor in the manufacture of a medicament for treating a cancer in a subject. Further provided is a pharmaceutical composition comprising a HIF-1α inhibitor for treating a cancer in a subject. The HIF-1α inhibitor may be intended to be used in combination with a second cancer immunotherapeutic agent.

The HIF-1α inhibitor may be echinomycin. The HIF-1α inhibitor may be used at a dose of about 100 to 1000 μg/m², as measured by body surface area (BSA). The second cancer immunotherapeutic agent may be an anti-CTLA-4 antibody, which may be Ipilimumab or Trememlimumab, or a derivative thereof. The HIF-1α inhibitor may target Tregs in the tumor microenvironment (TME). The HIF-1α may also abrogate PD-L1 in the TME, and may induce PD-L1 in normal tissues.

The treatment with the HIF-1α inhibitor and the anti-CTLA-4 antibody may exhibit improved safety as compared to combination cancer immunotherapy with an anti-PD-L1 antibody and the anti-CTLA-4 antibody. The improved safety may be fewer immune related adverse events, as measured in a population of subjects treated with the combination of the HIF-1α inhibitor and the anti-CTLA-4 antibody, as compared to a population of subjects treated with the anti-PD-L1 antibody and the anti-CTLA-4 antibody. In particular, the anti-CTLA-4 antibody may be Ipilimumab and the HIF-1α inhibitor may be echinomycin.

The cancer may be PD-L1-positive. The cancer may be characterized by significant infiltration of regulatory T-cells, and may be particularly amenable to immunotherapy with the HIF-1α inhibitor and the second cancer immunotherapeutic agent. The cancer may be a melanoma, lung cancer, non-small cell lung cancer, small cell lung cancer, squamous cell lung carcinoma, Hodgkin's lymphoma, classical Hodgkin's lymphoma, hairy leukemia, colorectal cancer, liver cancer, urothelial carcinoma, bladder cancer, renal cancer, renal cell carcinoma, kidney cancer, prostate cancer, head and neck squamous cell carcinoma, breast cancer, Merkel cell carcinoma, hepatocellular carcinoma, gastric cancer, advanced solid or hematologic malignancy, chronic lymphocytic leukemia, multiple myeloma, acute myeloid leukemia, MSI-high cancer, cervical cancer, mediastinal B-cell lymphoma, ovarian cancer, triple negative breast cancer, pancreatic cancer, glioblastoma, or medulloblastoma. The cancer characterized by significant infiltration of regulatory T-cells may in particular be a melanoma, non-small cell lung carcinoma, small cell lung cancer, squamous cell lung carcinoma, bladder cancer, renal cancer, breast cancer, liver cancer, pancreatic cancer, ovarian cancer, colorectal cancer, gastric cancer, or prostate cancer.

### BRIEF DESCRIPTION OF THE DRAWINGS

FIG. 1 shows a schematic of how HIF-1α inhibition by echinomycin abrogates PD-L1 expression in tumor tissues.

FIGS. 2A-J show that Hif-1α drives PD-L1 expression in tumor cells. FIG. 2A. Basal levels of Hif-1α protein in murine breast cancer cell lines as measured by Western blot. FIG. 2B. Effect of echinomycin on PD-L1 expression in 4T1 or E0771 cells. Tumor cells were treated with echinomycin (0.45 nM) or DMSO (vehicle) for 48 hours and then stained with anti-PD-L1 or isotype control, and analyzed by flow cytometry. Histograms for PD-L1 staining are shown. FIG. 2C. Effect of CoCl₂ on PD-L1 expression in E0771. E0771 cells were cultured as in FIG. 2B with CoCl₂ (250 μM) or PBS and then stained for PD-L1 and analyzed as in FIG. 2B. FIG. 2D. 4T1-HRE cells were treated for 24 hours with PBS or CoCl₂ (250 μM) prior to staining with PD-L1 or isotype control. The fluorescence intensity of EGFP is shown for each group. FIG. 2E. 4T1-HRE cells (1×10⁶), were orthotopically transplanted into BALB/c recipients (day 0). The mice were euthanized on day 21 and the tumor cells were stained for PD-L1, and analyzed by flow cytometry. Tumor cells (gated on Singlets/CD45⁻EGFP⁺) were further gated into top or bottom 30 percentiles (EGFP-high or low, respectively) and PD-L1 MFI minus isotype control was determined. Each dot represents mean fluorescence intensity of an independent tumor sample. Mean±SEM of each group of data, pooled from 3 independent experiments, are presented. FIG. 2F. Targeting Hif-1α suppresses PD-L1 in tumor tissues. 4T1 and E0771 (mammary fat pad), or MC38 cells (subcutaneous) were transplanted into BALB/c or C57BL/6 mice, which received vehicle or liposomal echinomycin (LEM, 0.25 mg/kg) every other day for 5 doses. Representative immunofluorescence images are shown, taken from the 4T1 (left), E0771 (middle), or MC38 (right) tumors of vehicle- (top) or echinomycin-treated (bottom) mice, two days after the final dose, and stained for PD-L1 (red) and DAPI (blue). FIGS. 2G-J. Effects of Hif-1α siRNA on PD-L1 expression in E0771 cells in vitro. E0771 cells with scrambled shRNA (sh⁻Scr), or knockdown of Hif1α (sh⁻Hif1α) were generated by lentiviral transduction and cultured under normoxia for 48 hours with DMSO (−), or echinomycin (1.35 nM, EM), stained with anti-PD-L1 or isotype control, and analyzed by flow cytometry. Representative histograms measuring PD-L1 intensity are shown in FIGS. 2G-I, comparing effects of targeted knockdowns (FIG. 2G), or the effects of echinomycin between sh⁻Scr (FIG. 2H) and sh⁻Hif1α (FIG. 2I) E0771 cells. The data are summarized in (FIG. 2J), expressed as mean±SEM of PD-L1 mean fluorescence intensity (MFI) for triplicate wells. Data are representative of three independent experiments.

FIGS. 3A-D show the therapeutic effects of echinomycin on tumor growth in immunodeficient and immunocompetent mice. FIG. 3A. Experimental design. Three murine tumor lines were tested: 4T1, E0771, or MC38. For each, immunodeficient and immunocompetent mice were inoculated (day 0), and treatment was initiated with control liposomes (vehicle) or echinomycin liposomes (LEM) on day 6, indicated by arrows in FIGS. 3B-3D. The kinetics of tumor growth were compared to deduce the role of adaptive immunity in the therapeutic effects of echinomycin. FIG. 3B. 4T1 cells (1×10⁶) were transplanted into mammary fat pad of NSG and BALB/c mice, which received vehicle or echinomycin (0.15 mg/kg) (arrows). Mean tumor volumes±SEM are shown for one of two independent experiments (n=10/group). FIG. 3C. Kinetics of E0771 tumor growth. E0771 cells (0.7×10⁶) were transplanted into mammary fat pad of NSG and C57BL/6 mice, which received vehicle or echinomycin (0.25 mg/kg) (arrows). Mean tumor volumes±SEM are shown for one of two independent experiments (n=5/group). FIG. 3D. Kinetics of MC38 tumor growth. MC38 cells (1×10⁶) were transplanted subcutaneously into NSG and C57BL/6 mice, which received vehicle or echinomycin (0.15 mg/kg) (blue arrows). Mean tumor volumes±SEM are shown for one of two independent experiments (n=5/group).

FIGS. 4A-D show the effects of pharmacological and/or genetic targeting of HIF-1α on E0771 tumor growth in immunodeficient or immunocompetent mice. FIG. 4A. Experimental design. Three sublines of E0771 were generated by lentiviral transduction: scrambled shRNA (sh⁻Scr), or shRNA for the Hif1α (sh⁻Hif1α) or Pdl1 (sh⁻Pdl1) genes. For each subline, 0.5×10⁶ cells were orthotopically transplanted into NSG or C57BL/6 mice (day 0), which received vehicle or echinomycin (0.25 mg/kg) starting day 6. FIG. 4B. Effects of Hif1α or Pdl1 knockdown on E0771 growth among immunodeficient or immunocompetent recipients. FIG. 4C. Effects of vehicle or echinomycin on sh⁻Scr, sh⁻Hif1α, or sh⁻Pdl1 E0771 growth in immunocompetent C57BL/6 recipients. FIG. 4D. Effects of vehicle or echinomycin (arrows) on sh⁻Scr, sh⁻Hif1α, or sh⁻Pdl1 E0771 growth in immunodeficient NSG recipients. Data shown are means+/−SEM (n=5/group) and are representative of 2 experiments.

FIGS. 5A-D show at echinomycin potentiates the therapeutic effect of anti-CTLA-4 antibody. FIG. 5A. Experimental design. In 4T1, E0771, or MC38 syngeneic tumor models, the effects of echinomycin on tumor growth were tested in combination with anti-CTLA-4 therapy (9D9). Tumor growth kinetics in mice receiving vehicle, echinomycin, and anti-CTLA-4 (9D9) monotherapies, or anti-CTLA-4+anti-PD-1 (9D9+RMP1-14) are shown. Starting on day 6 after tumor cell inoculation, mice received vehicle or echinomycin and/or various mAbs. The mean tumor volumes±SEM are shown for each group. FIG. 5B. Effects of 9D9+echinomycin on 4T1 tumor growth. BALB/c mice received vehicle, liposomal echinomycin (LEM, 0.15 mg/kg) (blue arrows), 9D9 (0.2 mg/mouse/injection) (red arrows), or both (n=10/group). Data shown for one of three independent experiments. FIG. 5C. Effects of 9D9+echinomycin on syngeneic E0771 growth. E0771 cells (0.5×10⁶) were orthotopically transplanted into the mammary fat pads of C57BL/6 mice. Mice received vehicle or echinomycin (0.25 mg/kg) and/or various mAbs (0.2 mg/mouse/injection) (red arrows). Representative data shown for one of three independent experiments (n=5/group). FIG. 5D. Effects of 9D9+echinomycin combination therapy on syngeneic MC38 growth. MC38 cells (1×10⁶) were transplanted into the left inguinal canal of C57BL/6 mice. Mice received vehicle or echinomycin (0.15 mg/kg) (blue arrows), and/or various mAbs (0.2 mg/mouse/injection) (red arrows). Representative data is shown for one of three independent experiments (n=5/group).

FIGS. 6A-G show that echinomycin suppresses PD-L1 on tumor cells and tumor-infiltrated myeloid cells and expands the IFNγ-producing CD8 and CD4 T cells regardless in the presence or absence of anti-CTLA-4 antibodies. FIGS. 6A-D. E0771 cells (0.5×10⁶) were orthotopically transplanted into C57BL/6 mice (day 0). The mice received treatment with vehicle, echinomycin (LEM, 0.25 mg/kg/dose), anti-CTLA-4 mAb (9D9, 0.2 mg/mouse/injection), or their combination on days 6, 8, 10, and 12, and were euthanized on day 14. The tumors were dissociated and analyzed by flow cytometry to quantitate PD-L1 expression on tumor cells (gated on Singlets/Live_CD45⁻) (FIG. 6A), M-MDSCs (gated on Singlets/Live_CD45⁺/CD11b⁺CD11c⁻/Ly6ChighLy6G⁻) (FIG. 6B), PMN-MDSCs (gated on Singlets/Live_CD45⁺/CD11b⁺CD11c⁻/Ly6CInt.Ly6G⁺) (FIG. 6C), or CD11c⁺TAMs (gated on Singlets/Live_CD45⁺/CD11b⁺CD11c⁺) (FIG. 6D). Upper panels show representative histograms of PD-L1 staining and lower panels show aggregate data from individual mice, pooled from three independent experiments (n=5 mice/group/experiment). Data shown are mean±SEM of PD-L1 MFI. Statistics were determined by one-way ANOVA with Sidak's multiple comparisons test (FIG. 6A), or by two-tailed unpaired t-tests (FIGS. 6B-D). FIGS. 6E-G. Echinomycin treatment reprograms the TME. Dissociated E0771 tumors were obtained from C57BL/6 mice treated with different therapies (n=5 mice/group), as described in FIGS. 6A-D. Single cell suspension were cultured for 4 hours with PMA+ionomycin+Golgi stop, surface stained, and then intracellular stained for IFNγ (for quantitation of Th1 and Tc1 cells) or surface stained for PD-L1. FIG. 6E. IFNγ expression in CD8+tumor-infiltrating lymphocytes (TILs) (gated on Singlets/Live_CD45⁺/CD3⁺/CD8⁺CD4⁻). The upper panels show representative histograms depicting IFNγ expression among different treatment groups and the lower panel shows mean±SEM of the frequencies of CD8⁺IFNγ⁺(Tc1) cells among the CD8⁺ TILs. FIG. 6F. IFNγ expression in the CD4⁺ TILs (gated on Singlets/Live_CD45⁺/CD3⁺/CD8⁻CD4⁺). As in FIG. 6E, except that CD4 TIL were analyzed.

FIG. 6G. MHCII expression on CD11c⁺ TAMs (gated on Singlets/Live_CD45⁺/CD11b⁺CD11c⁺). The upper panels show representative histograms depicting I-Ab expression among TAM, while the lower panel shows mean±SEM of MFI.

FIGS. 7A-H show that echinomycin stimulates PD-L1 expression in irAE target organs to limit the infiltration of T cell caused by anti-CTLA-4 mAbs by an IFNγ-dependent mechanism. E0771 cells (0.5×10⁶) were orthotopically transplanted into C57BL/6 mice (day 0), which were divided into 6 treatment groups: vehicle, echinomycin (LEM), anti-CTLA-4 (9D9), 9D9+echinomycin, 9D9+echinomycin+XMG1.2 (an anti-IFNγ antibody), or 9D9+RMP1-14. Echinomycin (0.25 mg/kg) or mAbs (0.2 mg/mouse/injection) were given on days 6, 8, 10, and 12. On day 14, the mice were euthanized, perfused, and the spleens were dissociated and analyzed by flow cytometry, or stimulated for 4 hours with PMA+ionomycin+GolgiStop prior to intracellular staining for IFNγ. The liver and kidney were processed for immunofluorescence analysis for PD-L1. FIG. 7A. PD-L1 expression in the tumor-bearing mice treated with different therapies. Representative immunofluorescence images shown for kidney (upper) and liver tissues (lower) of mice from indicated treatment groups stained by anti-PD-L1 and DAPI. FIGS. 7B-D. T cell infiltration in the liver and kidney of tumor-bearing mice. Representative immunofluorescence images from mice in the indicated treatment groups after staining with anti-CD3 and DAPI are shown in FIG. 7B for kidney (upper panel) and liver tissues (lower panel). T cell infiltration scores in the kidney (FIG. 7C) and liver (FIG. 7D) are shown as mean±SEM infiltration scores for the kidney (FIG. 7C) and liver tissues (FIG. 7D). T cell infiltration scoring range: 0, normal/none; 1, minimal; 2, mild; 3, moderate; 4, severe. FIG. 7E. Frequency of splenic Tc1 cells among CD8⁺ T cells (gated on Singlets/Live_CD45⁺/CD3⁺/CD8⁺CD4⁻), expressed as the mean±SEM. FIG. 7F. Frequencies of splenic Th1 cells among CD4⁺ T cells (gated on Singlets/Live_CD45⁺/CD3⁺/CD8⁻CD4⁺), expressed as the mean±SEM. FIG. 7G. Frequency of splenic T cells (gated on Singlets/Live_CD45⁺/CD3⁺/CD8⁺CD4⁻) among total hematopoietic cells (gated on Singlets/Live_CD45⁺). Representative data are shown for one of at least two independent experiments. FIG. 7H. CD3 and cleaved-caspase 3 staining in kidney and liver. Representative immunofluorescence images shown for kidney (upper) and liver tissues (lower) of mice that received 9D9 or 9D9+LEM. Caspase 3 positive cells are indicated with arrows. All data is representative of at least two independent experiments. In FIGS. 7C-G, data are presented as the mean±SEM with each dot representing an individual mouse, analyzed by one-way ANOVA with Sidak's posttest.

FIGS. 8A-H show that echinomycin induces PD-L1 to counter Ipilimumab-induced GI-irAEs by an IFNγ-dependent mechanism. FIGS. 8A-E. GI-irAE induction in human CTLA4 (CTLA4^{h/h}) knockin mice. FIG. 8A. Experimental design for GI-irAE induction by Ipilimumab, with or without additional therapies. 10-day old CTLA4h/h mice received 0.1 mg of Ipilimumab on days 10, 13, 16, and 19 after birth to induce the GI-irAE phenotype, measured by FITC-dextran assay on day 32. On day 33, the mice were sacrificed for histological and immunofluorescent analysis of intestinal tissues, and flow cytometry, etc. FIG. 8B. Representative immunofluorescence images showing relative infiltration of CD3+ T cells in jejunum of vehicle- (left) or Ipilimumab-treated (right) mice. FIG. 8C. Representative immunofluorescence images showing relative expression of PD-L1 in jejunum of vehicle-(left) or Ipilimumab-treated (right) mice. FIG. 8D. Association of intestinal PD-L1 expression with GI-irAE severity determined by FITC-dextran assay. The PD-L1 was detected in the intestines of Ipilimumab-treated mice and scored as negative/low (n=18) or high (n=21) based on the signal intensity level, and the serum FITC-dextran intensity is shown for each group. Aggregate data from 4 experiments are presented. FIGS. 8E-G. Effects of echinomycin, PD-1 blockade (RMP1-14) and IFNγ blockade (XMG1.2) on GI-irAEs in Ipilimumab-treated mice. Starting on day 10 after birth, the mice began receiving treatment with vehicle (n=33), echinomycin (n=27), Ipilimumab (n=33), Ipilimumab+echinomycin (n=26), Ipilimumab+RMP1-14 (n=27), Ipilimumab+echinomycin+RMP1-14 (n=28), or Ipilimumab+echinomycin+XMG1.2 (n=15). Echinomycin (10 μg/kg), Ipilimumab (0.1 mg/mouse/injection), RMP1-14 (0.2 mg/mouse/injection), and XMG1.2 (0.2 mg/mouse/injection). Vehicle or echinomycin (10 μg/kg) were given once every three days for 8 doses, and mAbs (0.1 mg/mo. for Ipilimumab; 0.2 mg/mo. for RMP1-14 and XMG1.2) once every three days for 4 doses. Data collection was performed as described in (FIG. 8A). FIG. 8E. Serum FITC-dextran intensity shown as mean±SEM for individual mice pooled from 3 independent experiments. Incidence of GI-irAEs corresponding to each group are shown as annotated percentages and the dotted line represents the threshold fluorescence intensity value for GI-irAE+. Statistics were determined by unpaired t tests (two-tailed) for group means. FIG. 8F. Representative H&E images from intestines of Ipilimumab-treated mice additionally treated with echinomycin (panel i), RMP1-14 (panels ii-iv), echinomycin+RMP1-14 (panels v-vi), or echinomycin+XMG1.2 (panels vii-viii). Panel i: duodenum, 20×, normal pathology; panel ii: duodenum, 20×, villous blunting and cell debris in lumen (arrows); panel iii: duodenum, 40×, with cellular debris in lumen; panel iv: duodenum, 40×, cellular debris and necrosis in lamina propria and epithelium; panel v: duodenum, 40×, debris and protein in lumen; panel vi: jejunum, 40×, debris outside cells in lumen; panel vii-viii: ileum, 40×, mild-moderate infiltration with lymphocytes, neutrophils, plasma cells, and mucosal mast cells. FIG. 8G. Representative immunofluorescence images showing relative expression of PD-L1 in jejunum of mice from different treatment groups. FIG. 8H. Flow cytometry analysis of PD-L1 expression in intestinal epithelial cells (gated on Singlets/Live/CD45-Cytokeratin+) from mice treated with Ipilimumab (n=6) or Ipilimumab+echinomycin (n=8). Data shown are means+/−SEM of the MFI.

FIGS. 9A-9I show that echinomycin improves TIL function in anti-CTLA-4 treated mice and CD8 TILs are critical for combination efficacy. FIGS. 9A-H. C57BL/6 mice received E0771 cells (0.5×10⁶/mouse) on day 0 followed by treatment with vehicle, echinomycin (LEM, 0.25 mg/kg/dose), anti-CTLA-4 (9D9, 0.2 mg/mouse/dose), or 9D9+LEM on days 6, 8, and 10. On day 14, tumors were analyzed by flow cytometry. Each graph shows the frequencies of CD8+ or CD4+subsets among CD8 or CD4 TILs (gated on Singlets/Live_CD45⁺/CD3⁺/CD8⁺CD4⁻, or Singlets/Live_CD45⁺/CD3⁺/CD8⁻CD4⁺, respectively). Data are presented as the ±SEM of each group (n=5/group), analyzed by unpaired two-tailed Student's t-test and are representative of two experiments. FIGS. 9A-B. Frequencies of TILs expressing PD-1. FIGS. 9C-D. Frequencies of annexin V⁺ CD8 or CD4 TILs. FIGS. 9E-F. Frequencies of CD8 TILs expressing granzyme B or perforin. FIGS. 9G-H. Granzyme B and perforin expression in CD4 TILs. FIG. 9I. Effect of depletion of CD4, CD8, or NK cells on tumor growth inhibition by 9D9+LEM in syngeneic E0771 model. C57BL/6 mice received E0771 cells (0.5×10⁶/mouse) on day 0. On day 5, the mice were randomized to receive depletory antibodies (500 μg of anti-CD4 (GK1.5), anti-CD8 (YTS169.4), anti-NK1.1 (PK136), or isotype ctrl). All groups received 9D9 (200 ug) on day 6, and LEM (250 ug/kg) on days 6, 8, and 10. Mice received supplemental dose of depletory antibodies (200 μg) on days 8 and 10. The mean±SEM tumor volumes are plotted on the y axes for each group (n=5/group) and analyzed by two-way ANOVA. Representative data shown for one of two experiments.

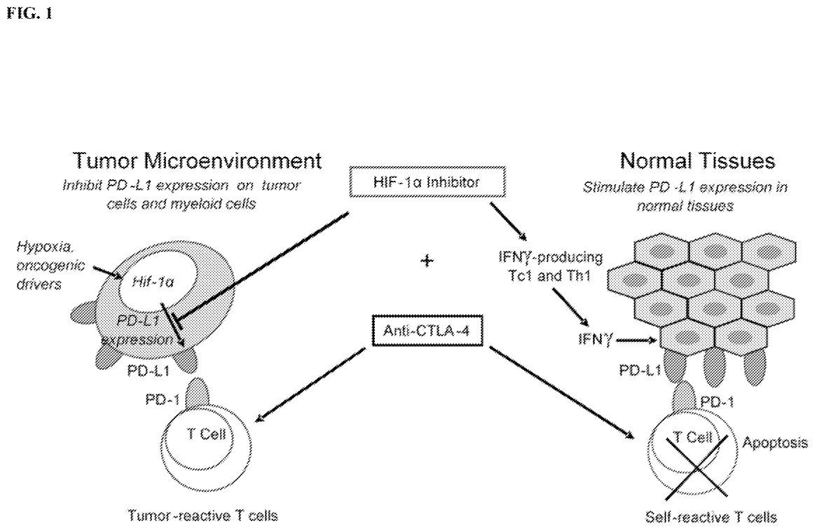

### DETAILED DESCRIPTION

A major limitation of anti-PD-1/PD-L1 mAbs is that they are incapable of distinguishing PD-1:PD-L1 interactions in the TME, which prevents effective cancer immunity, from PD-1:PD-L1 interactions in normal tissues, which protect against autoimmune diseases. Tumor-specific PD-L1 targeting would be more desirable as it may achieve cancer immunotherapy without causing irAE. This may be possible since the molecular mechanisms governing PD-L1 expression in normal tissues and cancer differ. For example, hypoxia, which is one of the major hallmarks distinguishing solid tumors from normal tissues (Muz et al., 2015), was reportedly responsible for inducing PD-L1 in tumor (Barsoum et al., 2014) and myeloid cells (Noman et al., 2014) via HIF-1α.

The inventors had the insight that Hif-1α inhibition may selectively repress PD-L1 expression in cancer. The inventors discovered that pharmaceutical or genetic targeting of Hif-1α suppresses PD-L1 expression in the TME, but paradoxically induces PD-L1 in normal tissues by enhancing T cell production of IFNγ. The data described herein demonstrate a new approach to differential regulation of PD-L1 for safer and more effective immunotherapy.

In particular, the combination of anti-CTLA-4 and anti-PD-1/PD-L1 antibodies is currently the most effective cancer immunotherapy, but it causes a high incidence of immune-related adverse events (irAE). The inventors have made the surprising discovery that HIF-1α inhibitors are as effective as anti-PD-L1 when used in cancer immunotherapy, but with fewer irAE. In particular, the inventors have discovered that targeting hypoxia-inducible factor 1α (HIF-1a) suppresses PD-L1 expression on tumor cells and tumor-infiltrated myeloid cells, but unexpectedly induces PD-L1 in normal tissues by an IFNγ-dependent mechanism. Targeting the HIF-1α-PD-L1 axis in tumor cells reactivates tumor-infiltrating lymphocytes (TILs) and causes tumor rejection. The HIF-1α inhibitor echinomycin potentiates cancer immunotherapeutic effects of anti-CTLA-4 therapy with efficacy comparable to anti-CTLA-4+anti-PD-1 antibodies. But while anti-PD-1 exacerbates irAE triggered by the anti-CTLA-4 antibody, Ipilimumab, echinomycin protects against irAEs by increasing PD-L1 levels in normal tissues. The inventors have further discovered that targeting HIF-1α fortifies the immune tolerance function of the PD-1:PD-L1 checkpoint in normal tissues but abrogates its immune evasion function in the tumor microenvironment to achieve safer and more effective immunotherapy.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Treatment” or “treating,” when referring to protection of an animal from a disease, means suppressing, repressing, reducing, or completely eliminating the disease. Suppressing the disease involves administering a composition of the present invention to an animal after induction of the disease but before its clinical appearance. Repressing the disease involves administering a composition of the present invention to an animal after clinical appearance of the disease. “Preventing” the disease involves administering a composition of the present invention to an animal prior to onset of the disease.

### 2. HIF-1α Inhibitors

Provided herein is an inhibitor of Hypoxia-Inducible Factor protein (HIF). The HIF inhibitor may be a HIF-1α inhibitor. The HIF inhibitor may be echinomycin, 2-methoxyestradiol, geldanamycin, CAY10585, chetomin, chrysin, dimethyloxaloylglycine, dimethyl-bisphenol A, PX 12, vitexin, or YC-1. In particular, the HIF-1α inhibitor may be echinomycin or an analog thereof. The HIF-1α inhibitor may also be a small interfering RNA (siRNA) or short hairpin RNA (shRNA) that targets HIF-1α and reduces or eliminates HIF-1α expression.

a. HIF

The HIF may be a functional hypoxia-inducible factor, which may comprise a constitutive b subset and an oxygen-regulated a subunit. The HIF may be over-expressed in a broad range of human cancer types, which may be a breast, prostate, lung, bladder, pancreatic or ovarian cancer. While not being bound by theory, the increased HIF expression may be a direct consequence of hypoxia within a tumor mass. Both genetic and environmental factors may lead to the increased HIF expression even under the normoxia condition. Germline mutation of the von Hippel-Lindau gene (VHL), which may be the tumor suppressor for renal cancer, may prevent degradation HIF under normoxia. It may be possible to maintain constitutively HIF activity under normoxia by either upregulation of HIF and/or down regulation of VHL. The HIF may be HIF1α or HIF2α.

Echinomycin and Analogs

Echinomycin (NSC526417) is a member of the quinoxaline family originally isolated from Streptomyces echinatus. Echinomycin is a small-molecule that inhibits the DNA-binding activity of HIF-1α. The echinomycin may be a peptide antibiotic such as N,N′-(2,4,12,15,17,25-hexamethyl-11,24-bis(1-methylethyl)-27-(methylthio)-3,6,10,13,16,19,23,26-octaoxo-9,22-dioxa-28-thia-2,5,12,15,18,25-hexaazabicyclo(12.12.3)nonacosane-7,20-diyl)bis(2-quinoxalinecarboxamide). The echinomycin may be a microbially-derived quinoxaline antibiotic, which may be produced by Streptomyces echinatus. The echinomycin may have the following structure.

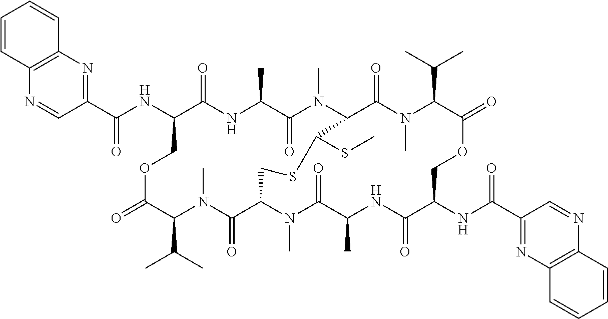 [SMILES: CSC1SC[C@H]2C(=O)N(C)[C@@H](C(C)C)C(=O)OC[C@@H](NC(=O)c3cnc4ccccc4n3)C(=O)N[C@@H](C)C(=O)N(C)[C@@H]1C(=O)N(C)[C@@H](C(C)C)C(=O)OC[C@@H](NC(=O)c1cnc3ccccc3n1)C(=O)N[C@@H](C)C(=O)N2C]

The echinomycin may have a structure as disclosed in U.S. Pat. No. 5,643,871, the contents of which are incorporated herein by reference. The echinomycin may also be an echinomycin derivative, which may comprise a modification as described in Gauvreau et al., Can J Microbiol, 1984; 30(6):730-8; Baily et al., Anticancer Drug Des 1999; 14(3):291-303; or Park and Kim, Bioorganic & Medicinal Chemistry Letters, 1998; 8(7):731-4, the contents of which are incorporated by reference. The echinomycin may also be a bis-quinoxaline analog of echinomycin.

Echinomycin analogues include compounds which due to their structural and functional similarity to echinomycin, exhibit effects on reduction of HIF-1α or HIF-2α activity, similar to that of echinomycin. Exemplary echinomycin analogues include YK2000 and YK2005 (Kim, J. B. et al., Int. J. Antimicrob. Agents, 2004 December; 24(6):613-615); Quinomycin G (Zhen X. et al., Mar. Drugs, 2015 Nov. 18; 13(11):6947-61); 2QN (Bailly, C. et al., Anticancer Drug. Des., 1999 June; 14(3):291-303); and quinazomycin (Khan, A. W. et al., Indian J. Biochem., 1969 December; 6(4):220-1).

b. Pharmaceutical Compositions

Also provided is a pharmaceutical composition comprising the HIF inhibitor and a pharmaceutically acceptable carrier. As used herein, the term “pharmaceutically acceptable” refers to a molecular entity or composition that does not produce an adverse, allergic or other untoward reaction when administered to an animal or a human, as appropriate. The term “pharmaceutically acceptable carrier,” as used herein, includes any and all solvents, dispersion media, coatings, antibacterial and/or antifungal agents, isotonic and absorption delaying agents, buffers, excipients, binders, lubricants, gels, surfactants and the like, that may be used as a media for a pharmaceutically acceptable substance. In one example, the pharmaceutical composition is a liposomal formulation.

Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, isotonic aqueous solutions, phosphate buffered saline, dextrose, 0.3% aqueous glycine, glycerol, ethanol and the like, as well as combinations thereof. In many cases, it will be preferable to include isotonic agents, for example, sugars, polyalcohols such as mannitol, sorbitol, or sodium chloride in the composition, or glycoproteins for enhanced stability, such as albumin, lipoprotein and globulin. Pharmaceutically acceptable carriers may further comprise minor amounts of auxiliary substances such as wetting or emulsifying agents, preservatives or buffers, which enhance the shelf life or effectiveness of the therapeutic agents.

These compositions can be sterilized by conventional sterilization techniques that are well-known to those of skill in the art. Sufficiently small liposomes, for example, can be sterilized using sterile filtration techniques.

Formulation characteristics that can be modified include, for example, the pH and the osmolality. For example, it may be desired to achieve a formulation that has a pH and osmolality similar to that of human blood or tissues to facilitate the formulation's effectiveness when administered parenterally. Alternatively, to promote the effectiveness of the disclosed compositions when administered via other administration routes, alternative characteristics may be modified.

Buffers are useful in the present invention for, among other purposes, manipulation of the total pH of the pharmaceutical formulation (especially desired for parenteral administration). A variety of buffers known in the art can be used in the present formulations, such as various salts of organic or inorganic acids, bases, or amino acids, and including various forms of citrate, phosphate, tartrate, succinate, adipate, maleate, lactate, acetate, bicarbonate, or carbonate ions. Particularly advantageous buffers for use in parenterally administered forms of the presently disclosed compositions in the present invention include sodium or potassium buffers, including sodium phosphate, potassium phosphate, sodium succinate and sodium citrate.

Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%).

In one embodiment, sodium phosphate is employed in a concentration approximating 20 mM to achieve a pH of approximately 7.0. A particularly effective sodium phosphate buffering system comprises sodium phosphate monobasic monohydrate and sodium phosphate dibasic heptahydrate. When this combination of monobasic and dibasic sodium phosphate is used, advantageous concentrations of each are about 0.5 to about 1.5 mg/ml monobasic and about 2.0 to about 4.0 mg/ml dibasic, with preferred concentrations of about 0.9 mg/ml monobasic and about 3.4 mg/ml dibasic phosphate. The pH of the formulation changes according to the amount of buffer used.

Depending upon the dosage form and intended route of administration it may alternatively be advantageous to use buffers in different concentrations or to use other additives to adjust the pH of the composition to encompass other ranges. Useful pH ranges for compositions of the present invention include a pH of about 2.0 to a pH of about 12.0.

In some embodiments, it will also be advantageous to employ surfactants in the presently disclosed formulations, where those surfactants will not be disruptive of the drug-delivery system used. Surfactants or anti-adsorbants that prove useful include polyoxyethylenesorbitans, polyoxyethylenesorbitan monolaurate, polysorbate-20, such as Tween-20TH, polysorbate-80, polysorbate-20, hydroxycellulose, genapol and BRIJ surfactants. By way of example, when any surfactant is employed in the present invention to produce a parenterally administrable composition, it is advantageous to use it in a concentration of about 0.01 to about 0.5 mg/ml.

Additional useful additives are readily determined by those of skill in the art, according to particular needs or intended uses of the compositions and formulator. One such particularly useful additional substance is sodium chloride, which is useful for adjusting the osmolality of the formulations to achieve the desired resulting osmolality. Particularly preferred osmolalities for parenteral administration of the disclosed compositions are in the range of about 270 to about 330 mOsm/kg. The optimal osmolality for parenterally administered compositions, particularly injectables, is approximately 3000 sm/kg and achievable by the use of sodium chloride in concentrations of about 6.5 to about 7.5 mg/ml with a sodium chloride concentration of about 7.0 mg/ml being particularly effective.

Echinomycin-containing liposomes or echinomycin-containing microemulsion drug-delivery vehicles can be stored as a lyophilized powder under aseptic conditions and combined with a sterile aqueous solution prior to administration. The aqueous solution used to resuspend the liposomes can contain pharmaceutically acceptable auxiliary substances as required to approximate physical conditions, such as pH adjusting and buffering agents, tonicity adjusting agents and the like, as discussed above.

In other embodiments the echinomycin-containing liposomes or echinomycin-containing microemulsion drug-delivery vehicle can be stored as a suspension, preferable an aqueous suspension, prior to administration. In certain embodiments, the solution used for storage of liposomes or microemulsion drug carrier suspensions will include lipid-protective agents which protect lipids against free-radical and lipid-peroxidative damage on storage. Suitable protective compounds include free-radical quenchers such as alpha-tocopherol and water-soluble iron-specific chelators, such as ferrioxamine.

The HIF inhibitor may be formulated as described in U.S. Patent Application Publication No. 2018/0344642, the contents of which are incorporated herein by reference. In one example, the HIF inhibitor is formulated as a liposomal drug formulation, which may include a peglyated phospholipid, a neutral phosphoglyceride, and a sterol. The PEGylated liposomes may encapsulate the HIF inhibitor. The PEGylated phospholipid may be one or more of distearoylphosphatidylethanolamine-polyethylene glycol (DSPE-PEG), a dimyristoyl phosphatidylethanolamine-polyethylene glycol (DMPE-PEG), a dipalmitoylglycerosuccinate polyethylene glycol (DPGS-PEG), a cholesteryl-polyethylene glycol, and a ceramide-based pegylated lipid. The neutral phosphoglyceride may be one or more of phosphatidylcholine, phosphatidylserine, phosphatidylethanolamine, phosphatidylglycerol and phosphatidylinositol. The molar ratio of the PEGylated phospholipid to total lipids in the formulation may be from 3 to 6%. The molar ratio of the neutral phosphoglyceride to total lipids in the formulation may be from 45 to 65%. The molar ratio of the sterol to total lipids in the formulation may be from 30 to 50%. In one example, the PEGylated phospholipid is a distearoylphosphatidylethanolamine-polyethylene glycol (DSPE-PEG), the neutral phosphoglyceride is a phosphatidylcholine, and the sterol is cholesterol. The formulation may include DSPE-PEG-2000, hydrogenated soybean phosphatidylcholine (HSPC), and cholesterol. In one example, the molar ratios of DSPE-PEG-2000, HSPC, and cholesterol to total lipids are 5.3%, 56.3%, and 38.4%, respectively. The mass ratio of HIF inhibitor to total lipids may be from 2 to 10%. In one example, the mass ratio of HIF inhibitor to total lipids is 5%. In another example, at least 90% of the liposomes in the formulation have a diameter between 80 and 120 nm. In particular, the HIF inhibitor may be echinomycin.

The pharmaceutical composition may be in the form of tablets or lozenges formulated in a conventional manner. For example, tablets and capsules for oral administration may contain conventional excipients may be binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers may be lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants may be potato starch and sodium starch glycollate. Wetting agents may be sodium lauryl sulfate. Tablets may be coated according to methods well known in the art.

The pharmaceutical composition may also be liquid formulations such as aqueous or oily suspensions, solutions, emulsions, syrups, and elixirs. The pharmaceutical composition may also be formulated as a dry product for constitution with water or other suitable vehicle before use. Such liquid preparations may contain additives such as suspending agents, emulsifying agents, nonaqueous vehicles and preservatives. Suspending agents may be sorbitol syrup, methyl cellulose, glucose/sugar syrup, gelatin, hydroxyethylcellulose, carboxymethyl cellulose, aluminum stearate gel, and hydrogenated edible fats. Emulsifying agents may be lecithin, sorbitan monooleate, and acacia. Nonaqueous vehicles may be edible oils, almond oil, fractionated coconut oil, oily esters, propylene glycol, and ethyl alcohol. Preservatives may be methyl or propyl p-hydroxybenzoate and sorbic acid.

The pharmaceutical composition may also be formulated as suppositories, which may contain suppository bases such as cocoa butter or glycerides. The pharmaceutical composition may also be formulated for inhalation, which may be in a form such as a solution, suspension, or emulsion that may be administered as a dry powder or in the form of an aerosol using a propellant, such as dichlorodifluoromethane or trichlorofluoromethane. Agents provided herein may also be formulated as transdermal formulations comprising aqueous or nonaqueous vehicles such as creams, ointments, lotions, pastes, medicated plaster, patch, or membrane.

The pharmaceutical composition may also be formulated for parenteral administration such as by injection, intratumor injection or continuous infusion. Formulations for injection may be in the form of suspensions, solutions, or emulsions in oily or aqueous vehicles, and may contain formulation agents including, but not limited to, suspending, stabilizing, and dispersing agents. The pharmaceutical composition may also be provided in a powder form for reconstitution with a suitable vehicle including, but not limited to, sterile, pyrogen-free water.

The pharmaceutical composition may also be formulated as a depot preparation, which may be administered by implantation or by intramuscular injection. The pharmaceutical composition may be formulated with suitable polymeric or hydrophobic materials (as an emulsion in an acceptable oil, for example), ion exchange resins, or as sparingly soluble derivatives (as a sparingly soluble salt, for example).

c. Administration

Administration of the HIF inhibitor or pharmaceutical composition thereof may be orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular. For veterinary use, the agent may be administered as a suitably acceptable formulation in accordance with normal veterinary practice. The veterinarian can readily determine the dosing regimen and route of administration that is most appropriate for a particular animal. The pharmaceutical composition may be administered to a human patient, cat, dog, large animal, or an avian.

In certain embodiments, the composition can be formulated as a depot preparation. Such long acting formulations may be administered by implantation at an appropriate site or by parenteral injection, particularly intratumoral injection or injection at a site adjacent to cancerous tissue.

When the HIF inhibitor is encapsulated in a liposome or other microemulsion drug-delivery vehicle, any effective amount of the echinomycin or echinomycin may be administered. Preferably, the liposomal formulations or other microemulsion drug-delivery vehicles containing echinomycin, an echinomycin derivative, or an echinomycin analogue are administered by parenteral injection, including intravenous, intraarterial, intramuscular, subcutaneous, intra-tissue, intranasal, intradermal, instillation, intracerebral, intrarectal, intravaginal, intraperitoneal, intratumoral.

Intravenous administration of liposomal echinomycin has been tolerated by mice at doses of approximately 1 mg/kg of body weight and no LD50 value has been reached. In contrast, free echinomycin has an LD50 value of 0.629 mg/kg.

Liposomal preparations or other microemulsion delivery vehicles can be lyophilized and stored as sterile powders, preferably under vacuum, and then reconstituted in bacteriostatic water (containing, for example, benzyl alcohol preservative) or in sterile water prior to injection. Pharmaceutical compositions may be formulated for parenteral administration by injection e.g., by bolus injection or continuous infusion.

The delivery vehicle may be administered to the patient at one time or over a series of treatments and may be administered to the patient at any time from diagnosis onwards. The delivery vehicle may be administered as the sole treatment or in conjunction with other drugs or therapies useful in treating the condition in question.

The pharmaceutical composition may be administered simultaneously or metronomically with other treatments. The term “simultaneous” or “simultaneously” as used herein, means that the pharmaceutical composition and other treatment be administered within 48 hours, preferably 24 hours, more preferably 12 hours, yet more preferably 6 hours, and most preferably 3 hours or less, of each other. The term “metronomically” as used herein means the administration of the agent at times different from the other treatment and at a certain frequency relative to repeat administration.

The pharmaceutical composition may be administered at any point prior to another treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins, 10 mins, 9 mins, 8 mins, 7 mins., 6 mins., 5 mins., 4 mins., 3 mins, 2 mins, and 1 mins. The pharmaceutical composition may be administered at any point prior to a second treatment of the pharmaceutical composition including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

The pharmaceutical composition may be administered at any point after another treatment including about 1 min, 2 mins., 3 mins., 4 mins., 5 mins., 6 mins., 7 mins., 8 mins., 9 mins., 10 mins., 15 mins., 20 mins., 25 mins., 30 mins., 35 mins., 40 mins., 45 mins., 50 mins., 55 mins., 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, 48 hr, 50 hr, 52 hr, 54 hr, 56 hr, 58 hr, 60 hr, 62 hr, 64 hr, 66 hr, 68 hr, 70 hr, 72 hr, 74 hr, 76 hr, 78 hr, 80 hr, 82 hr, 84 hr, 86 hr, 88 hr, 90 hr, 92 hr, 94 hr, 96 hr, 98 hr, 100 hr, 102 hr, 104 hr, 106 hr, 108 hr, 110 hr, 112 hr, 114 hr, 116 hr, 118 hr, and 120 hr. The pharmaceutical composition may be administered at any point prior after a pharmaceutical composition treatment of the agent including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

d. Dosage

The pharmaceutical composition may be administered in a therapeutically effective amount of the HIF inhibitor to a mammal in need thereof. The therapeutically effective amount required for use in therapy varies with the nature of the condition being treated, the length of time desired to inhibit HIF activity, and the age/condition of the patient.

HIF inhibitor dosages can be tested in a suitable animal model as further described below. As a general proposition, a therapeutically effective amount of HIF inhibitor or other anti-cancer agent will be administered in a range from about 10 ng/kg body weight/day to about 100 mg/kg body weight/day whether by one or more administrations. In a particular embodiment, each therapeutic agent is administered in the range of from about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 μg/kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In one example, the HIF inhibitor is administered at a body surface area (BSA)-based dose of 10-30,000 μg/m², 100-30,000 μg/m², 500-30,000 μg/m², 1000-30,000 μg/m², 1500-30,000 μg/m², 2000-30,000 μg/m², 2500-30,000 μg/m², 3000-30,000 μg/m², 3500-30,000 μg/m², 4000-30,000 μg/m², 100-20,000 μg/m², 500-20,000 μg/m², 1000-20,000 μg/m², 1500-20,000 μg/m², 2000-20,000 μg/m², 2500-20,000 μg/m², 3000-20,000 μg/m², 3500-20,000 μg/m², 100-10,000 μg/m², 500-10,000 μg/m², 1000-10,000 μg/m², 1500-10,000 μg/m², 2000-10,000 μg/m², or 2500-10,000 μg/m².

In one example, the HIF inhibitor is echinomycin. The echinomycin may be administered at a dose of about 100-1000 μg/m², or about 100, 150, 200, 250, 300, 350, 400, 450, 500, 550, 600, 650, 700, 750, 800, 850, 900, 950, or 1000 μg/m². The echinomycin may be administered 1, 2, or 3, particularly 3, times a week. The echinomycin may be administered over a period of 4-10 weeks, or about 4, 5, 6, 7, 8, 9, or 10 weeks. The echinomycin may be injected intravenously. The dose regimen may achieve optimal therapeutic effect, which may occur without significant adverse effects.

In other embodiments, the HIF inhibitor is administered in the range of about 10 ng to about 100 ng per individual administration, about 10 ng to about 1 μg per individual administration, about 10 ng to about 10 μg per individual administration, about 10 ng to about 100 μg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 μg per individual administration, about 100 ng to about 10 μg per individual administration, about 100 ng to about 100 μg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 μg to about 10 μg per individual administration, about 1 μg to about 100 μg per individual administration, about 1 μg to about 1 mg per individual administration, about 1 μg to about 10 mg per individual administration, about 1 μg to about 100 mg per individual administration, about 1 μg to about 1000 mg per injection, about 1 μg to about 10,000 mg per individual administration, about 10 μg to about 100 μg per individual administration, about 10 μg to about 1 mg per individual administration, about 10 μg to about 10 mg per individual administration, about 10 μg to about 100 mg per individual administration, about 10 μg to about 1000 mg per injection, about 10 μg to about 10,000 mg per individual administration, about 100 μg to about 1 mg per individual administration, about 100 μg to about 10 mg per individual administration, about 100 μg to about 100 mg per individual administration, about 100 μg to about 1000 mg per injection, about 100 μg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The fusion protein or expression vector may be administered daily, every 2, 3, 4, 5, 6 or 7 days, or every 1, 2, 3 or 4 weeks.

In other particular embodiments, the amount of HIF inhibitor may be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage will be dependent on the condition, size, age and condition of the patient.

The therapeutic agents in the pharmaceutical compositions may be formulated in a “therapeutically effective amount.” A “therapeutically effective amount” refers to an amount effective, at dosages and for periods of time necessary, to achieve the desired therapeutic result. A therapeutically effective amount of the liposomal formulation or other microemulsion drug-delivery vehicle may vary depending on the condition to be treated, the severity and course of the condition, the mode of administration, the bioavailability of the particular agent(s), the ability of the delivery vehicle to elicit a desired response in the individual, previous therapy, the age, weight and sex of the patient, the patient's clinical history and response to the antibody, the type of the fusion protein or expression vector used, discretion of the attending physician, etc. A therapeutically effective amount is also one in which any toxic or detrimental effects of the delivery vehicle is outweighed by the therapeutically beneficial effects.

The HIF inhibitor dose may be a non-toxic dose. The dose may also be one at which HIF activity is inhibited, but at which c-Myc activity is unaffected. In general, however, doses employed for adult human treatment typically may be in the range of 1-100 μg/m² per day, or at a threshold amount of 1-100 μg/m² per day or less, as measured by a body-surface adjusted dose. The desired dose may be conveniently administered in a single dose, or as multiple doses administered at appropriate intervals, for example as two, three, four or more sub-doses per day. Multiple doses may be desired, or required.

The dosage may be a dosage such as about 1 μg/m², 2 μg/m², 3 μg/m², 4 μg/m², 5 μg/m², 6 μg/m², 7 μg/m², 8 μg/m², 9 μg/m², 10 μg/m², 15 μg/m², 20 μg/m², 25 μg/m², 30 μg/m², 35 μg/m², 40 μg/m², 45 μg/m², 50 μg/m², 55 μg/m², 60 μg/m², 70 μg/m², 80 μg/m², 90 μg/m², 100 μg/m², 200 μg/m², 300 μg/m², 400 μg/m², 500 μg/m², 600 μg/m², 700 μg/m², 800 μg/m², 900 μg/m², 1000 μg/m², 1100 μg/m², or 1200 μg/m², and ranges thereof.

The dosage may also be a dosage less than or equal to about 1 μg/m², 2 μg/m², 3 μg/m², 4 μg/m², 5 μg/m², 6 μg/m², 7 μg/m², 8 μg/m², 9 μg/m², 10 μg/m², 15 μg/m², 20 μg/m², 25 μg/m², 30 μg/m², 35 μg/m², 40 μg/m², 45 μg/m², 50 μg/m², 55 μg/m², 60 μg/m², 70 μg/m², 80 μg/m², 90 μg/m², 100 μg/m², 200 μg/m², 300 μg/m², 400 μg/m², 500 μg/m², 600 μg/m², 700 μg/m², 800 μg/m², 900 μg/m², 1000 μg/m², 1100 μg/m², or 1200 μg/m², and ranges thereof.

### 3. Methods of Cancer Immunotherapy

Provided herein is a method of treating a cancer. The treatment may be a cancer immunotherapy. The method may comprise administering the HIF inhibitor to a subject in need thereof. The subject may be a mammal, which may be a human patient. Also provided herein is a composition comprising the HIF inhibitor for use in treating the cancer, or use of the HIF inhibitor in the manufacture of a medicament for treating cancer.

a. Combination therapy

The HIF inhibitor may be used alone, or in combination with a second anti-cancer therapy in a method or use described herein. The HIF inhibitor may be used in combination with one or more anti-cancer immunotherapies. In particular, the anti-cancer immunotherapy may be an anti-CTLA-4 antibody. Anti-CTLA4 antibodies are known in the art. The anti-CTLA-4 may be Ipilimumab or Tremelimumab, or a mutant form or derivative thereof. The wild-type or mutant anti-CTLA-4 antibody may be described in International Publication WO/2017/106372 or WO/2019/152423, the contents of which are incorporated herein by reference.

The anti-cancer immunotherapy used in combination with the HIF inhibitor may include one or more anti-PD-1 antibodies, which may be pembrolizumab (Keytruda®), nivolumab (Opdivo®), tislelizumab, toripalimab, or camrelizumab. The combination may also include one or more anti-PD-L1 (anti-B7-H1) antibodies, which may be atezolizumab (Tecentriq®) durvalumab, avelumab, or cemplimab. In another example, the HIF inhibitor may be combined with one or more of an anti-B7-H3 antibody, anti-B7-H4 antibody, anti-LIGHT antibody, anti-LAG3 antibody, anti-TIM3 antibody, anti-TIM4 antibody, anti-CD40 antibody, anti-OX40 antibody, anti-GITR antibody, anti-BTLA antibody, anti-CD27 antibody, anti-ICOS antibody, or anti-4-1BB antibody. The HIF inhibitor may be administered in combination with molecules that activate different stages or aspects of the immune response in order to achieve a broader immune response.

In another example, the anti-cancer immunotherapy may involve one or more molecules that disrupt or enhance alternative immunomodulatory pathways (such as TIM3, TIM4, OX40, CD40, GITR, 4-1-BB, PD-L1, PD-1, B7-H3, B7-H4, LIGHT, BTLA, ICOS, CD27 or LAG3) or modulate the activity of effecter molecules such as cytokines (e.g., IL-4, IL-7, IL-10, IL-12, IL-15, IL-17, GF-beta, IFNg, Flt3, BLys) and chemokines (e.g., CCL21) in order to enhance the immunomodulatory effects.

b. Cancer

“Cancer,” as used herein, may refer to a neoplasm or tumor resulting from abnormal uncontrolled growth of cells. “Cancer” explicitly includes leukemias and lymphomas. The term “cancer” also refers to a disease involving cells that have the potential to metastasize to distal sites.

In particular, the cancer may be a tumor. In one example, the cancer may be positive for PD-L1. That is, the cancer may express PD-L1, which may be at high levels. It is known in the art how to determine whether a cancer expresses PD-L1, and particularly high levels of PD-L1. Methods of measuring PD-L1 expression in cancers are known in the art. The PD-L1-positive cancer may be identified by using one or more of immunostaining, Western blotting, quantitative polymerase chain reaction (qPCR), or microarray. The PD-L1-positive cancer may be melanoma, lung cancer, non-small cell lung cancer, small cell lung cancer, squamous cell lung carcinoma, Hodgkin's lymphoma, classical Hodgkin's lymphoma, hairy leukemia, colorectal cancer, liver cancer, urothelial carcinoma, bladder cancer, renal cancer, renal cell carcinoma, kidney cancer, prostate cancer, head and neck squamous cell carcinoma, breast cancer, Merkel cell carcinoma, hepatocellular carcinoma, gastric cancer, advanced solid or hematologic malignancy, chronic lymphocytic leukemia, multiple myeloma, acute myeloid leukemia, MSI-high cancer, cervical cancer, mediastinal B-cell lymphoma, ovarian cancer, triple negative breast cancer, pancreatic cancer, glioblastoma, or medulloblastoma. The cancer may also be a cancer known to be treatable with anti-PD-1/anti-PD-L1 immunotherapy.

The cancer may be a cancer that may be treatable with anti-CTLA-4 antibodies, particularly when the HIF inhibitor is used in combination with an anti-CTLA-4 antibody. In particular, the cancer may be a cancer with significant infiltration of regulatory T cells. The cancer may be a cancer described herein. In one example, the cancer is a melanoma (including metastatic), non-small cell lung carcinoma, small cell lung cancer, squamous cell lung carcinoma, bladder cancer, renal cancer, breast cancer, liver cancer, pancreatic cancer, ovarian cancer, colorectal cancer, gastric cancer, bladder cancer, or a prostate cancer such as metastatic hormone-refractory prostate cancer.

The methods and compositions described herein may also be useful in the treatment or prevention of one or more of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include, but are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. In other specific embodiments, sarcoma, melanoma, or leukemia is treated or prevented by the methods and compositions described herein.

The present invention has multiple aspects, illustrated by the following non-limiting examples.

### Example 1

### HIF-1α Inhibitors are Effective in Cancer Immunotherapy

This example demonstrates that HIF-1α inhibition, including the use of HIF-1α inhibitors, is an effective cancer immunotherapy.

Targeting Hif-1α Suppresses PD-L1 Expression in TME

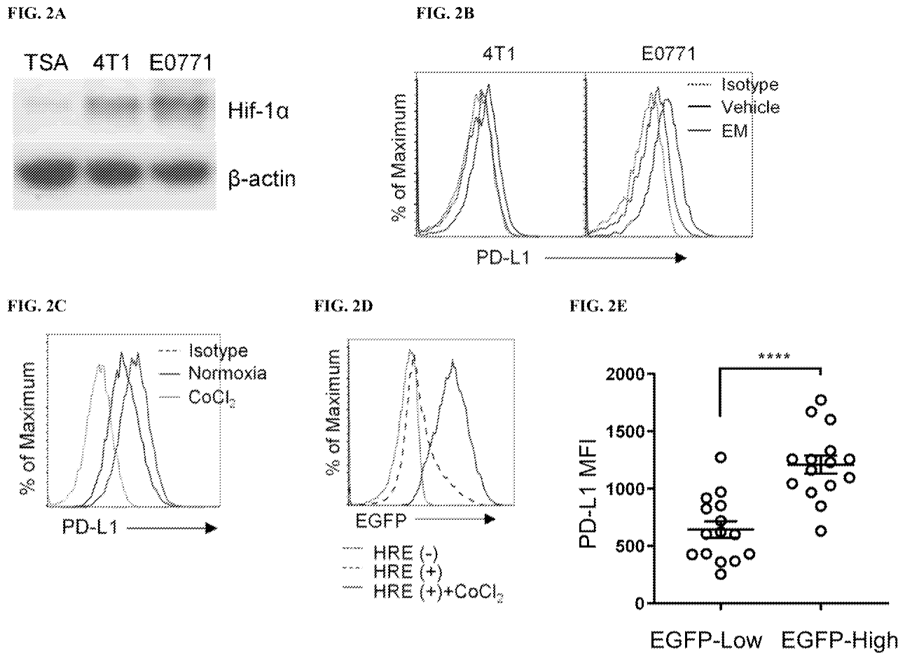

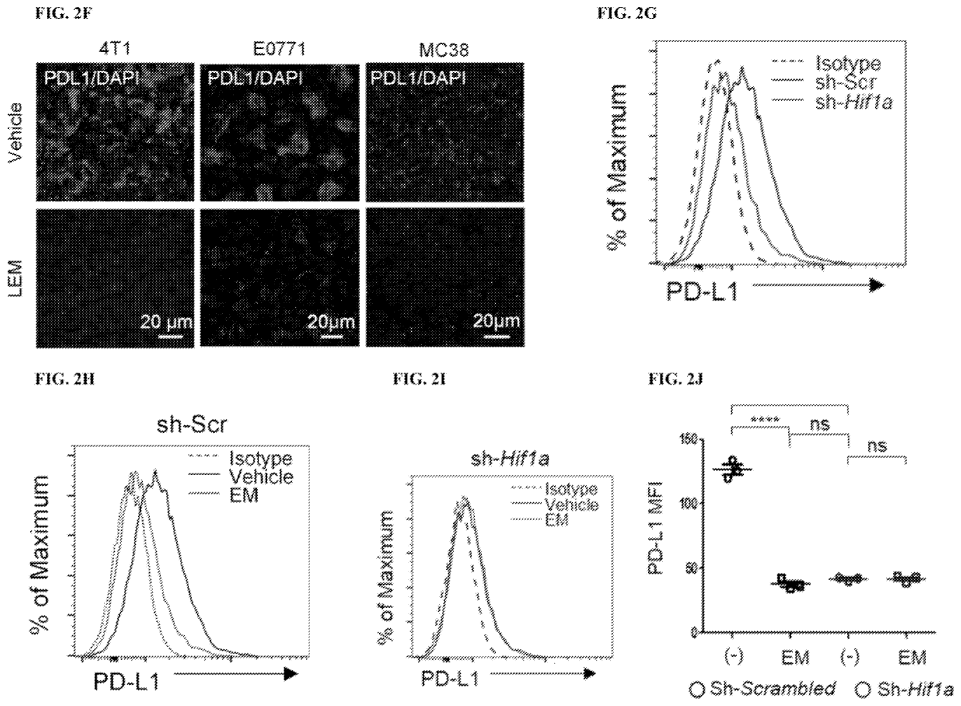

Previous studies have shown that hypoxia induces PD-L1 through transcriptional activation of PD-L1 transcription by HIF-1α. Since tumor cells also express HIF-1α under normoxia, we tested if the Hif-1α-PD-L1 axis is also active in tumor cells expressing stable Hif-1α under normoxic conditions. We first examined levels of Hif-1α and PD-L1 in various murine tumor cell lines cultured under normoxia. In 4T1 and E0771 breast cancer cell lines, we confirmed the co-expression of Hif-1α and PD-L1 by immunoblot (FIG. 2A) and flow cytometry (FIG. 2B), and treatment of either cell line with the Hif-1α inhibitor echinomycin reduced PD-L1 protein (FIG. 2B). Consistent with previous reports of hypoxia-induced PD-L1 expression (Barsoum et al., 2014), treatment of E0771 cells with hypoxia mimetic CoCl₂ further upregulated PD-L1 from the basal levels seen at normoxia (FIG. 2C). To demonstrate the relationship between Hif-1α activity and PD-L1 protein expression in tumor cells in vivo, we transduced 4T1 cells with a lentiviral transcription factor reporter construct containing the core HRE motif upstream of an EGFP reporter. In response to CoCl₂ stimulation, the resultant 4T1-HRE-EGFP cells exhibited a marked increase in EGFP reporter fluorescence activity (FIG. 2D). After engrafting the 4T1-HRE-EGFP cells into immunocompetent BALB/c mice and allowing solid tumors to form, we analyzed PD-L1 expression on the isolated tumor cells by flow cytometry. PD-L1 expression was associated with EGFP reporter activity in the isolated tumor cells (FIG. 2E). The results suggest that an Hif-1α may regulate PD-L1 expression on tumor cells in vivo. To test this hypothesis, we evaluate the effect of echinomycin on intratumoral PD-L1 expression by immunofluorescence staining of PD-L1 for the fixed tumor specimens from engrafted tumor cell lines. As shown in FIG. 2F, there was a marked reduction in PD-L1 expression in the tumors of echinomycin-treated mice.

To demonstrate that the pharmacologic inhibition of Hif-1α by echinomycin is the mechanism responsible for reduction of PD-L1 protein, we used siRNA to knockdown Hif1α in E0771 cells and quantified PD-L1 expression by flow cytometry after 24-hour incubation with vehicle or echinomycin (FIG. 2G-J). Under basal conditions, we found that knockdown of Hif1α reduced PD-L1 protein expression (FIG. 2G, I, J). Moreover, while the inhibitory effect of echinomycin on PD-L1 expression was preserved in E0771 cells that received scrambled shRNA (FIG. 2H-J) knockdown of Hif1α abrogated the ability for echinomycin to decrease PD-L1 protein (FIG. 2H-J). These results demonstrate that Hif-1α controls PD-L1 expression in E0771 cells. Moreover, since knockdown of Hif1α also abrogated PD-L1 response to echinomycin, echinomycin reduces PD-L1 by inhibiting the Hif-1α-PD-L1 axis.

Immunotherapeutic Effect of Echinomycin

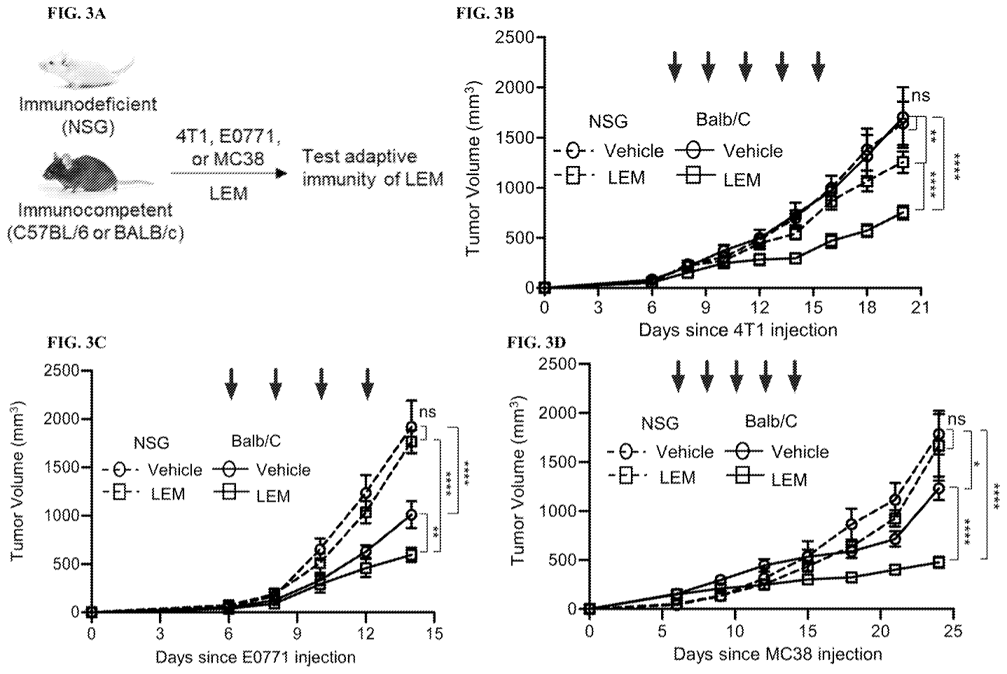

Given the profound effect of PD-L1 on immune function, it was of interest to test if Hif-1α inhibition results in an immunotherapeutic effect on cancer. To address this, we first compared the therapeutic effects of pharmacological Hif-1α inhibition with echinomycin on tumor outgrowth in mice sufficient or deficient in adaptive immunity (FIG. 3A). Echinomycin significantly inhibited 4T1 tumor growth in both immunocompetent (BALB/c) and immunodeficient (NSG) recipients, as compared to each strain's respective vehicle control (FIG. 3B). However, 4T1 growth was more significantly inhibited in immunocompetent mice than in immunodeficient mice, which suggested an immunotherapeutic effect of echinomycin in addition to potentially tumor-intrinsic therapeutic effects in this model. In a second breast cancer model, E0771, we observed therapeutic effect on immune competent but not in immunodeficient mice, which suggested that the effect in the immunocompetent hosts are largely due to immunotherapy (FIG. 3C). To test if Hif-1α inhibition can confer an immunotherapeutic effect in a non-breast cancer model, we repeated the experiments using MC38 murine colon adenocarcinoma cells. As with E0771, all therapeutic effects required immune competence (FIG. 3D).

Echinomycin Inhibits PD-L1 in Tumor Cells by Targeting the Hif-1α-PD-L1 Axis

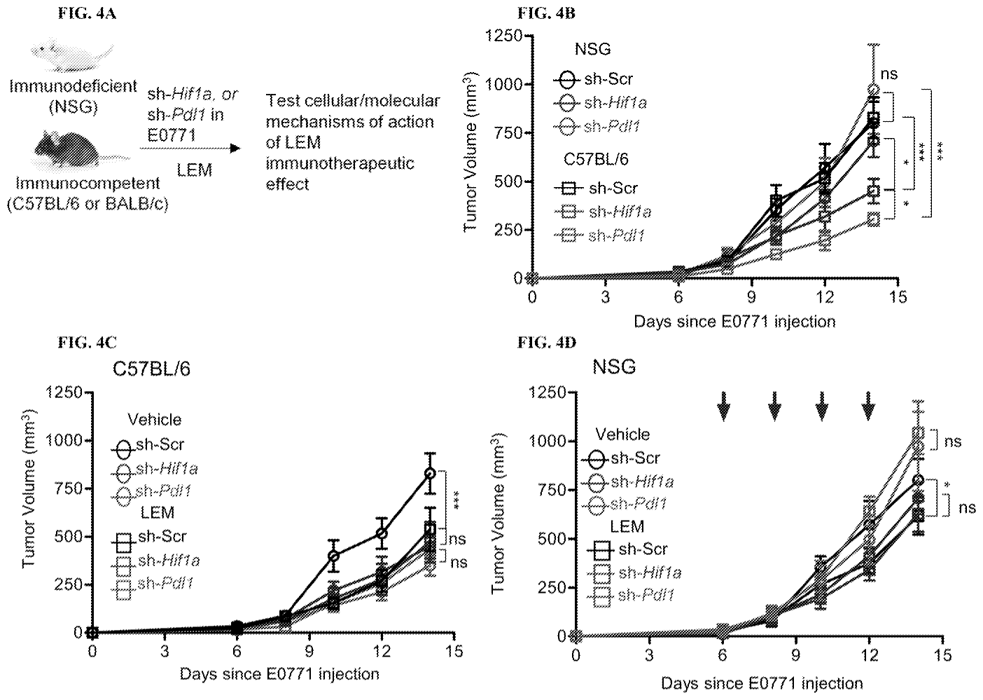

To further investigate the cellular and molecular mechanisms of action of echinomycin with respect to its immunotherapeutic effect and inhibitory effects on intratumoral PD-L1 expression, we compared the effects of targeted knockdown of Hif1α or Pd1 in E0771 cells on the tumor growth kinetics among immunocompetent or immunodeficient recipient strains. In parallel, we treated both strains of recipient mice with vehicle or echinomycin to measure the impact of tumor cell-intrinsic Hif1α or Pdl1 on tumor growth therapeutic response to echinomycin (FIG. 4A). In C57BL/6, but not NSG recipients, genetic depletion of Hif1α (sh-Hif1α) in E0771 cells significantly inhibited tumor growth compared to E0771 transduced with scrambled shRNA (sh⁻Scr) in mice of the same respective strains (FIG. 4B). Moreover, the tumor growth rates of E0771 with Hif1α knockdown were also significantly reduced in immunocompetent vs immunodeficient recipients (FIG. 4B). As in FIG. 3C, echinomycin more effectively inhibited sh⁻Scr E0771 tumor growth in C57BL/6 (FIG. 4C), compared to NSG (FIG. 4D) recipients; in contrast, echinomycin did not inhibit sh⁻Hif1α E0771 tumor growth, regardless of the recipient strain (FIGS. 4C and D). Thus, pharmacologic or genetic targeting of Hif-1α in tumor cells alone can confer an immunotherapeutic effect. Furthermore, the loss of biological activity for echinomycin with respect to tumor growth kinetics following knockdown of Hif1α in E0771 provides genetic evidence that echinomycin confers an immunotherapeutic effect in vivo by targeting the Hif-1α in tumor cells.

In the same manner, we next analyzed the effects of Pdl1 knockdown to determine whether downregulation of PD-L1 is critical in the immunotherapeutic effect of echinomycin. Much like the knockdown of Hif1α, genetic depletion of Pdl1 (sh⁻Pdl1) in E0771 cells also inhibited tumor growth in C57BL/6 but not NSG recipients (FIG. 4B), and echinomycin did not further suppress sh-Pdl1 E0771 tumor growth in C57BL/6 (FIG. 4C) or NSG (FIG. 4D) recipients. Taken together, the data support the conclusion that echinomycin confers immunotherapeutic effects in vivo by targeting the Hif-1α-PD-L1 axis in tumor cells.

Hif-1α Inhibition Potentiates Anti-CTLA-4 Immunotherapy

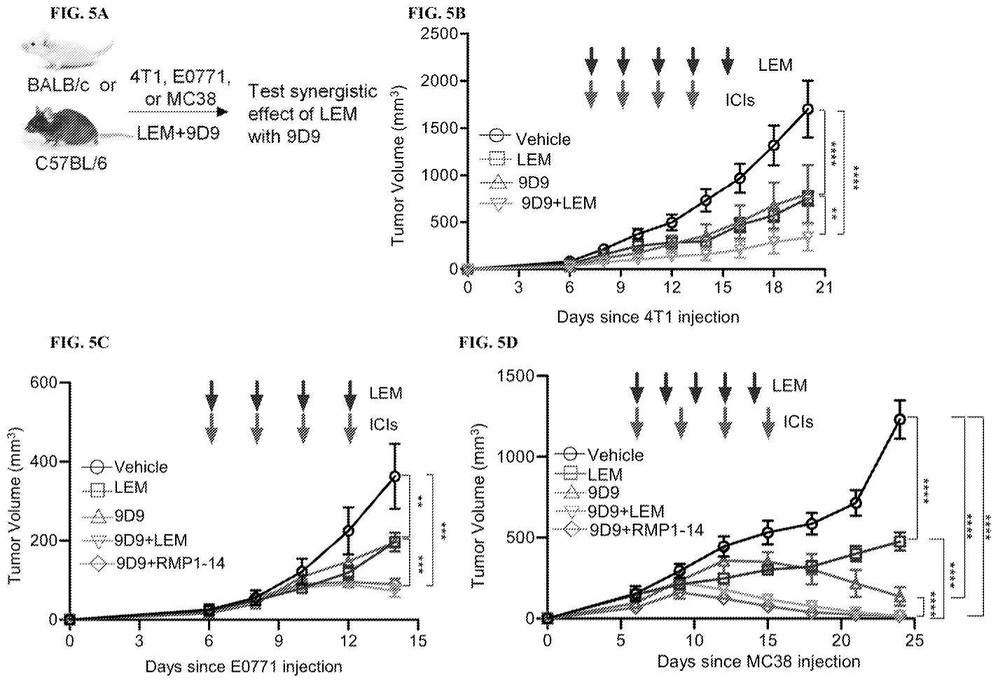

Co-targeting targeting CTLA-4 and PD-1/PD-L1 immune checkpoints simultaneously with their respective blocking mAbs is the most efficacious strategy currently available for cancer immunotherapy. Having established that echinomycin can target PD-L1 in tumor cells and promote an immunotherapeutic effect in vivo, we explored whether this strategy may also potentiate immunotherapeutic effects in the context of anti-CTLA-4 therapy. We examined the therapeutic effects of CTLA-4 blocking mAbs, with or without echinomycin, using 4T1, E0771, or MC38 syngeneic mouse models of cancer (FIG. 5A). As shown in FIG. 5B, anti-mouse CTLA-4 mAb (9D9) in combination with echinomycin significantly inhibited 4T1 tumor growth more effectively than either monotherapy. To further investigate the combination efficacy of targeting Hif-1α during anti-CTLA-4 therapy, we performed similar drug treatment experiments using immunocompetent C57BL/6 recipients and the E0771 breast cancer (FIG. 5C), or MC38 colon adenocarcinoma (FIG. 5D) models and observed synergistic effect in all models. We also compared the effects on combination with echinomycin to that with PD-1 mAb (RMP1-14). Again, we observed significant inhibition of tumor growth by echinomycin or 9D9 monotherapies compared to vehicle, while the greatest inhibition was achieved by 9D9+echinomycin or RMP1-14 (FIGS. 5C and D). These data demonstrated therapeutic effect of blocking PD-1:PD-L1 interaction can be similarly achieved by either anti-PD-1 or echinomycin.

Echinomycin Inhibits PD-L1 on Tumor Cells and Tumor-Infiltrated Myeloid Cells

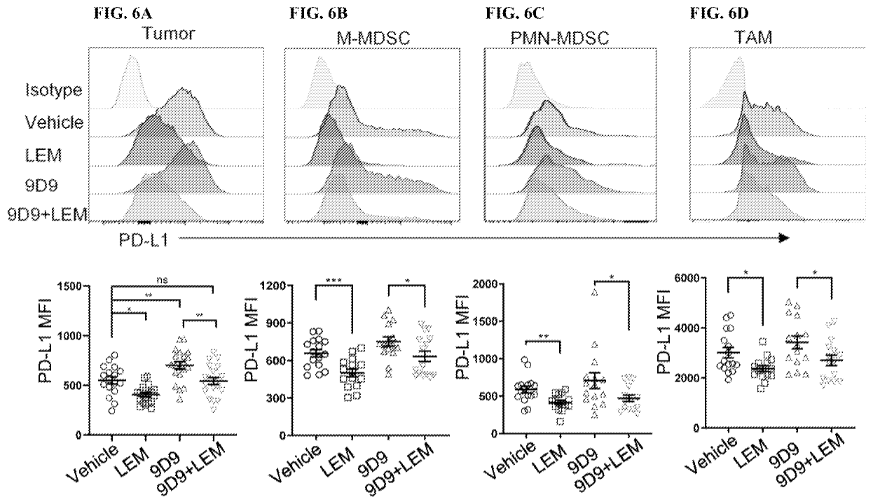

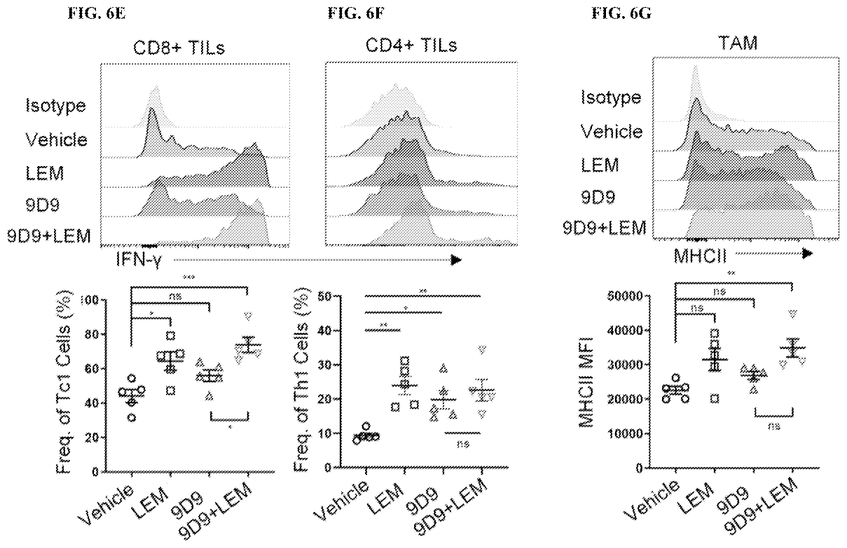

We have shown systemic Hif-1α inhibition suppressed PD-L1 expression in multiple tumors (FIG. 2F). To gain insight as to the cellular landscape and cell-specific expression patterns of PD-L1 in the TME following echinomycin and/or 9D9 treatment, we analyzed E0771 tumors from C57BL/6 mice treated with vehicle, echinomycin, 9D9, or 9D9+echinomycin for the composition of immune cells. While Echinomycin did not significantly impact the frequencies of tumor-infiltrated lymphocytes or myeloid subsets, 9D9 reduced the frequencies of polymorphonuclear MDSCs (PMN-MDSCs). However, echinomycin significantly reduced PD-L1 expression on tumor cells (FIG. 6A), and tumor-infiltrated monocytic MDSCs (M-MDSC) (FIG. 6B), PMN-MDSCs (FIG. 6C), and CD11b⁺CD11c⁺ double-positive cells (FIG. 6D), with or without anti-CTLA-4 therapy. Further analysis revealed that the overwhelming majority of cells in the CD11b⁺CD11c⁺ subset were TAMs, as roughly 90% of the CD11b⁺CD11c⁺ cells co-expressed F4/80, consistent with the earlier report (Gordon et al., 2017). The results show that, in addition to tumor cells, in vivo HIF-1α inhibition can also suppress PD-L1 on tumor-infiltrated myeloid cells, and these effects persist in the context of anti-CTLA-4 therapy. More importantly, the results provide evidence supporting the notion that Hif-1α plays a vital role in coordinating PD-L1 expression on various tumor-infiltrated immune subsets in the TME.

To test whether Hif-1α inhibition can rescue the function of TILs in the TME, we used flow cytometry to measure the frequencies of IFNγ-expressing CD3⁺ TILs after culturing the single-cell suspensions from dissociated E0771 tumors in the presence of PMA+ionomycin. Compared to vehicle, all treatments increased the frequencies of both IFNγ⁺CD8⁺ (Tc1) and IFNγ⁺CD4⁺ (Th1) subsets, although the highest frequencies of either were observed in mice receiving 9D9+echinomycin (FIGS. 6E and F). MHCII expression also increased on CD11b⁺CD11c⁺ cells in response to the treatments in a pattern strikingly similar to that of the frequencies of Tc1 TILs, thus reflecting the individual or combined contributions of the therapies in promoting tumor inflammation (FIG. 6G).

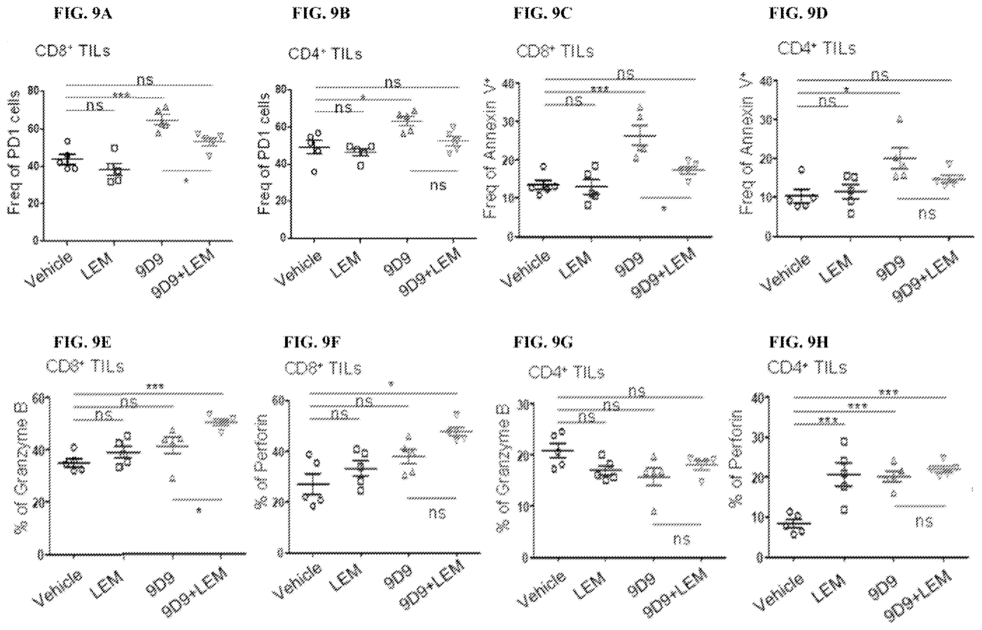

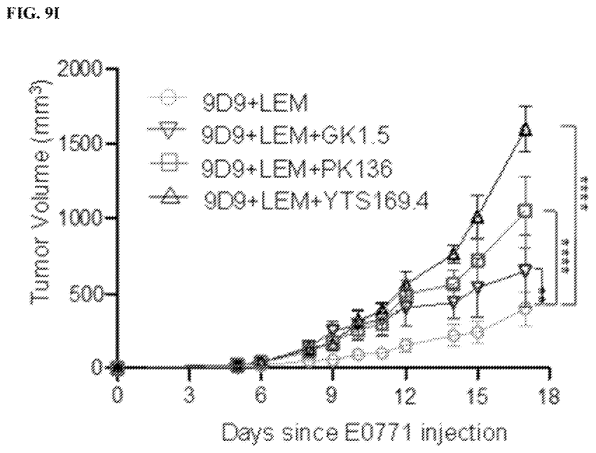

To better understand the impact of pharmacologic Hif-1α targeting in the context of immunotherapy, we performed more detailed analysis of TILs. E0771 mice treated with anti-CTLA-4 had higher expression of exhaustion marker PD-1 on CD8 TILs compared to vehicle, which was reversed by echinomycin (FIG. 9A). The same was seen for PD-1 expression on CD4 TILs (FIG. 9B) In addition to TIL exhaustion, PD-L1 can also induce TIL apoptosis. Annexin V staining revealed more apoptotic CD8 and CD4 TILs in tumors of 9D9-treated mice compared to vehicle and adding echinomycin appeared to repress this effect in CD8 TILs (FIG. 9C). The same trend was seen for CD4 TILs, although the difference between 9D9 and 9D9+LEM groups was not significant (FIG. 9D). Higher expression of cytolytic effector molecules granzyme B and perforin were noted in CD8 TILs of 9D9+LEM treated mice vs vehicle (FIGS. 9E and F). Roughly one fifth of CD4 TILs were granzyme B+, which was not significantly affected by drug treatments (FIG. 9G). On the other hand, in all treated groups, the mean frequencies of CD4 TILs expressing perforin roughly doubled that of the control group (FIG. 9H).

We used depletory antibodies to assess the impact of CD4, CD8, and NK cells in the combinational efficacy of 9D9+LEM in E0771 mice. These studies revealed that optimal efficacy required all three cell types, with CD8 being the most critical, followed by NK and CD4 cells (FIG. 9I). Thus, the immunotherapeutic effects of pharmacological Hif-1α inhibition in context of anti-CTLA-4 are multi-cell dependent, but primarily depend on CD8 T cells.

Echinomycin alone inhibited PD-L1 on tumor cells and tumor-infiltrating myeloid cells but increased the proportion of CD8 TILs expressing IFNγ (FIG. 6). An important question arose as to whether echinomycin improves CD8 TIL function directly by a T cell-intrinsic mechanism, or indirectly through reducing PD-L1 on tumor and/or myeloid cells. To test this, we generated mice with conditional knockout of Hif1α in T lineages using the cre lox system. Loss of Hif1α did not significantly impact the proportion of CD4 and CD8 TILs expressing IFNγ, T-bet, or RORγt, or the frequencies of Tregs or Th17. In CD4, but not CD8 TILs, we found an increased frequency of PD-1⁺ cells. Granzyme B and perforin in CD8 TILs was slightly reduced in Hif1α KO mice, but not significantly. In contrast, knockdown of Pdl1 in the tumor cells significantly increased the frequencies of Tc1 and Th1 cells, phenocopying the effects of echinomycin. Therefore, suppression of PD-L1 on tumor cells can at least partially account for the enhanced CD8 TIL function and therapeutic effects provided by echinomycin in the immune competent mouse. Notably, inhibition of PD-L1 on tumor cells by echinomycin was preserved in mice with conditional knockout of Hif1α in T cells. These data indicate that the decreased PD-L1 expression is not due to an T cell-intrinsic effect echinomycin.

It has been reported that TILs tend to avoid hypoxic zones in the TME. Using known methods, we noted an increase in CD3 TIL infiltration into hypoxic areas of the tumors in echinomycin-treated mice.

Echinomycin Induces PD-L1 Expression to Limit Anti-CTLA-4-Induced T Cell Infiltration in irAE Target Organs

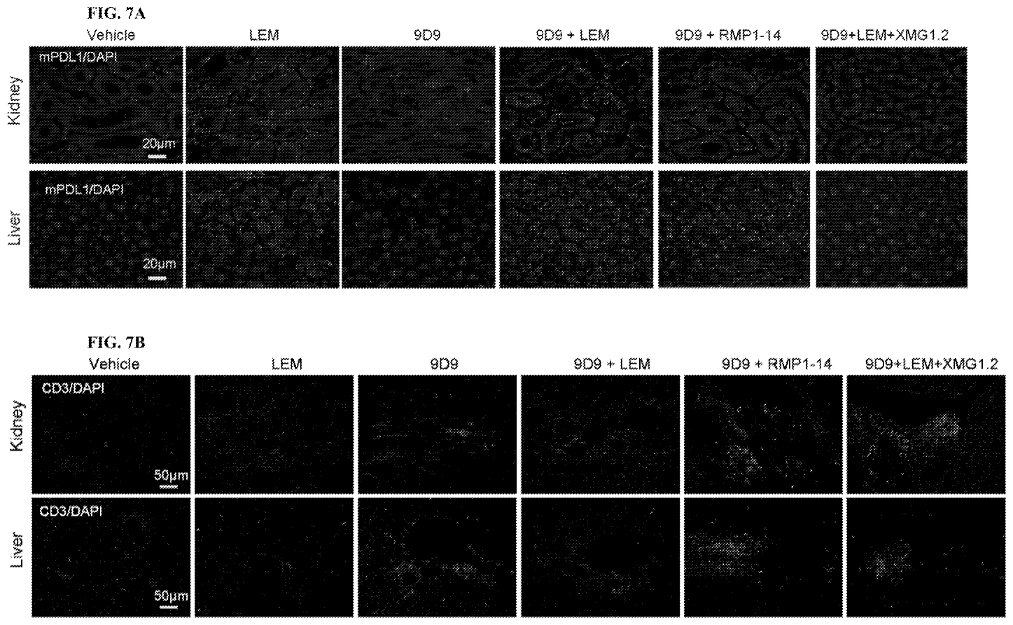

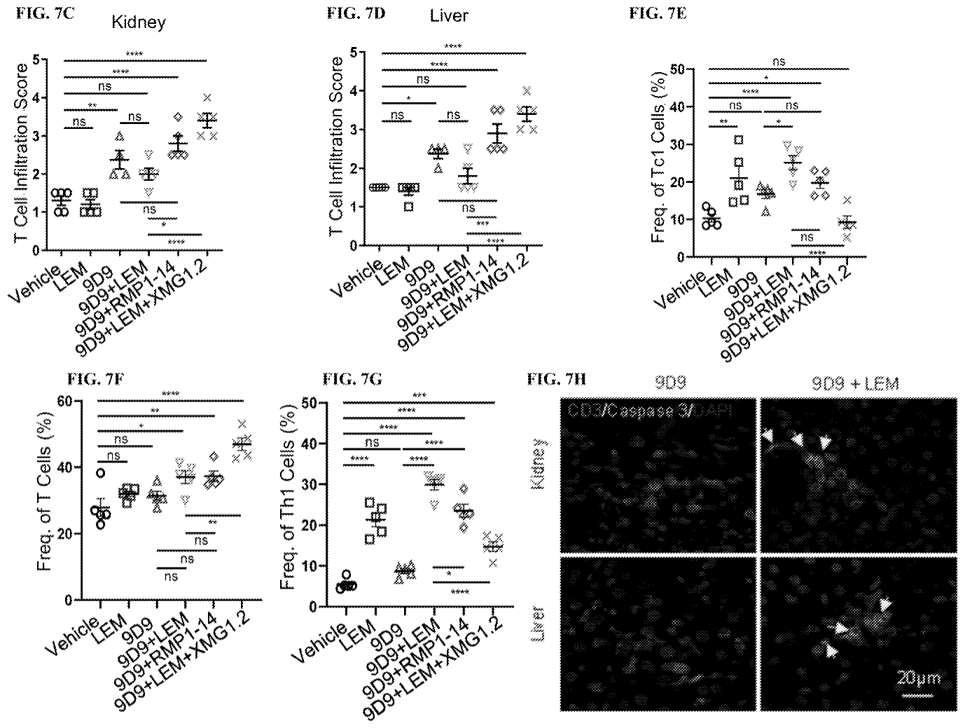

To test if PD-L1 is induced on the tissue level in response to anti-CTLA-4 therapy, we performed immunofluorescence staining of PD-L1 and CD3 in the liver and kidney of tumor-bearing mice treated with 9D9 alone or in combination with other therapies (FIG. 7A). PD-L1 expression in these tissues was elevated in mice treated with 9D9 (FIG. 7A). Interestingly, echinomycin also induced PD-L1 (FIG. 7A), but only 9D9 resulted in hepatic and renal infiltration of T cells (FIGS. 7B-D). T cell infiltration was reduced in 9D9+LEM treated mice when compared to 9D9 alone (FIGS. 7B-D). In contrast, the frequency of T cells, as well as the frequencies Tc1 and Th1, expanded in the spleen of mice that received 9D9+echinomycin treatment (FIGS. 7E-G). Thus, the reductions of T cells in liver and kidney by echinomycin were not the result of general T cell inactivation. Rather, cleaved-caspase 3 staining suggested that the induced PD-L1 regulates T infiltration by triggering apoptosis (FIG. 7H). When 9D9 was combined with anti-PD-1 (RMP1-14), the mice had high intensity of T cell infiltration in liver and kidney (FIGS. 7B-D). The frequencies of T cells in the spleen, including Tc1 and Th1, were comparable when either anti-PD-1 or echinomycin were used in conjunction with anti-CTLA-4 mAb (FIGS. 7E-G). Since IFNγ is known to upregulate PD-L1 in normal tissues, we hypothesized PD-L1 induction by echinomycin could be due to IFNγ. Echinomycin alone did not stimulate increased infiltration of T or NK cells in the tissues with PD-L1 expression (FIGS. 7B-D).

We further tested the importance of IFNγ by using the anti-IFNγ neutralizing mAb, XMG1.2, which abrogated PD-L1 induction by 9D9+echinomycin treatment and increased T cell infiltration in the kidneys and liver (FIGS. 7B-D). XMG1.2 also abrogated PD-L1 expression in the kidney and liver in absence of 9D9, indicating that IFNγ is responsible for PD-L1 induction by LEM in these tissues. Conditional knockout of Hif1α in T cells did not phenocopy the effects of echinomycin on PD-L1 induction in the liver, but PD-L1 induction was preserved regardless of the mouse genotype. To test whether LEM can reduce irAE in the adult tumor-bearing mouse, we measured serum biomarkers for hepatic, renal, and gastrointestinal irAE. However, the adult mouse tolerated high dose of anti-CTLA-4 antibody without significant irAE.

Echinomycin Protected Ipilimumab-Induced irAEs in Human CTLA4-Knockin Mice

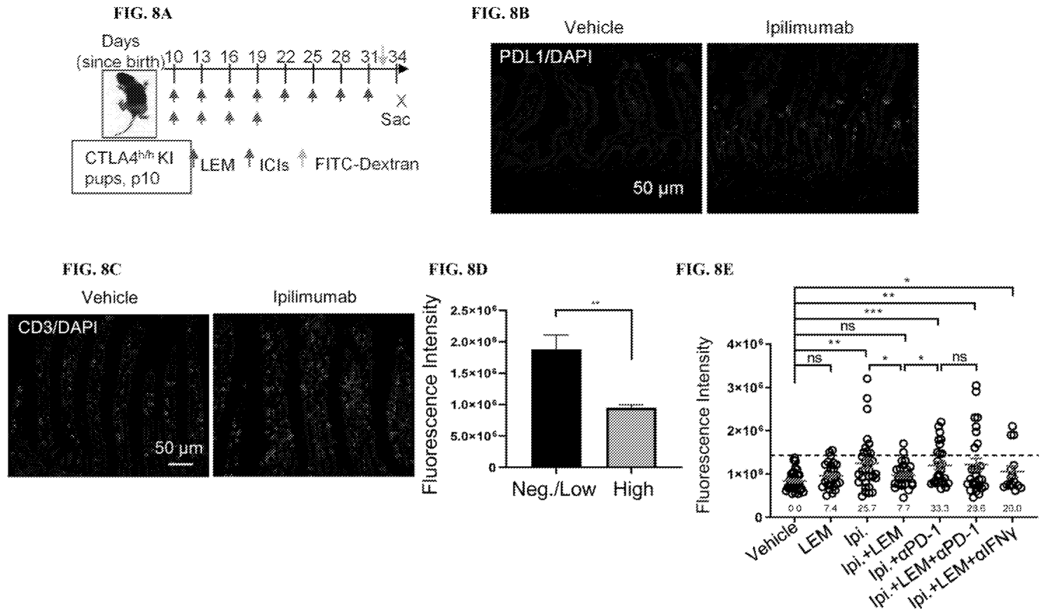

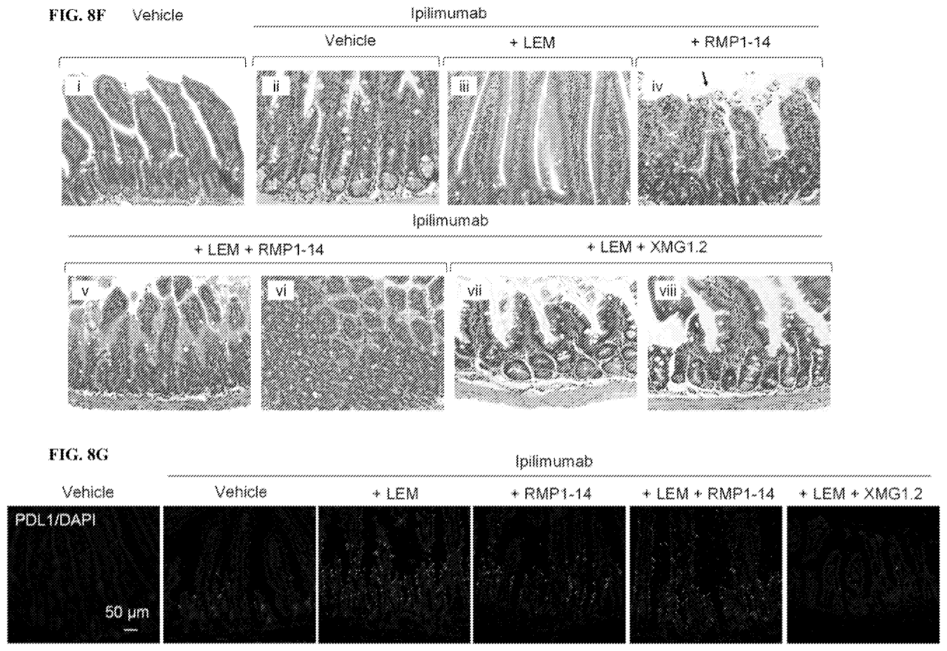

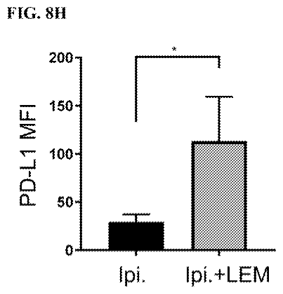

Gastrointestinal track is the most frequent target of irAEs (Luoma et al., 2020). Therefore, we used intestinal permeability to orally administrated FITC-dextran and histology as the readout for irAE (FIG. 8A). As what was described for liver and kidney, Ipilimumab treatment result in elevated PD-L1 expression (FIG. 8B) and T cell accumulation (FIG. 8C) in the intestines. To explore whether PD-L1 could serve a functional role in the protection from GI-irAEs induced by Ipilimumab, we compared the fluorescence intensity of FITC-dextran measured in the sera among those with high or low PD-L1 staining in the intestines. We observed that mice with high levels of intestinal PD-L1 had much lower intestinal permeability (FIG. 8D). The association between intestinal permeability and PD-L1 expression supports the hypothesis that Ipilimumab-induced PD-L1 serves as a limiting factor against Ipilimumab-induced GI-irAEs.

To test this hypothesis, we assessed whether blockade of PD-1:PD-L1 checkpoint during Ipilimumab treatment would also worsen GI-irAEs in the CTLA4 knockin model, and how this approach might compare to substitution of anti-PD-1 mAbs with echinomycin. We evaluated % of mice with significantly higher serum FITC-dextran than control mice, using means+2 SD as boundary for intestinal leakage. As shown in FIG. 8E, echinomycin protected against Ipilimumab-induced intestinal leakage by a PD-L1-dependent mechanism as this protection is abrogated by anti-PD-1 (FIG. 8E). Moreover, in mice that received Ipilimumab+echinomycin treatment, addition of anti-IFNγ antibody increased the frequency of mice with intestinal leakage from 7.7% to 20.0% (FIG. 8E). Collectively, the data suggested that through induction of IFNγ, echinomycin confers protection against Ipilimumab-induced GI-irAEs by elevating PD-L1 expression to fortify the PD-1:PD-L1 checkpoint.

To further investigate PD-L1 expression in the intestinal tissues in response to Ipilimumab and to validate its role in conferring protection from Ipilimumab-induced GI-irAEs, we performed histological analysis of the intestinal tissue and immunofluorescence staining of PD-L1. Consistent the FITC-dextran data in FIG. 8E, Ipilimumab induced intestinal inflammation. The inflammation is largely abrogated by echinomycin as mice treated with Ipilimumab+echinomycin for the most part exhibited normal intestinal pathology (FIG. 8F). Echinomycin enhanced PD-L1 expression in the intestine compared to Ipilimumab alone (FIG. 8G). The elevated PD-L1 expression is confirmed by flow cytometry using digested intestinal tissues (FIG. 8H).

To confirm the significance of induced PD-L1 in protection against inflammation in the intestine, we used anti-PD-1 mAb to block PD-1/PDL-1 interaction. These data shown that the protective effect of echinomycin is abrogated by the anti-PD-1 antibodies (FIG. 8F). Moreover, IFNγ was elevated in intestine from mice that received treatment of both echinomycin and Ipilimumab, and the protective effect of echinomycin depends on IFNγ as the effect was abolished by the anti-IFNγ mAb XMG1.2 (FIG. 8F). These data suggested that the IFNγ-PD-L1 axis is responsible for the echinomycin-mediated protection against Ipilimumab-induced GI-irAE.

Discussion

HIF-1α inhibition is an area of active investigation in cancer therapy (Peng and Liu, 2015; Semenza, 2003). We have reported that echinomycin effectively eliminated leukemia stem cells (Wang et al., 2011). However, clinical development of echinomycin for solid tumor has met with minimal success. In our studies of breast cancer, we found that reformulating echinomycin with liposomes enabled potent therapeutic effects in orthotopic xenograft mouse models of triple-negative breast cancer (TNBC), including primary tumor growth and metastasis in the MDA-MB-231 and SUM-159 models (Bailey et al., 2020). The current study supports echinomycin's re-emergence as an immunotherapeutic agent.

The pioneering work in developing immunotherapy targeting PD-1 and PD-L1 (Dong et al., 2002; Iwai et al., 2002; Iwai et al., 2005; Strome et al., 2003; Wang et al., 2014) has led to the most important breakthrough in cancer therapy, with rapidly expanding indications of anti-PD1/PD-L1 antibodies adopted for treatment of both hematological and non-hematological malignancies (Sanmamed and Chen, 2019). However, the current approach that overcomes tumor evasion of host immunity also disables the immune tolerance checkpoint, leading to significant irAEs, particularly when used in conjunction with anti-CTLA-4 antibodies. Here, we showed that targeting HIF-1α not only overcomes immune evasion in the TME, but also fortifies the immune tolerance checkpoint in normal tissues.

HIF-1α is generally inactivated in normal tissues but frequently stabilized in tumor cells regardless of oxygen tension (Iommarini et al., 2017; Talks et al., 2000). This fundamental difference allows us to selectively inhibit PD-L1 expression in the tumor microenvironment using echinomycin. Surprisingly, echinomycin induced PD-L1 expression in normal tissues of immunocompetent mice, including liver, kidney, salivary gland and colon. The unexpected induction of PD-L1 was attributable to elevated IFNγ production associated with echinomycin-induced expansion of IFNγ-producing T cells, including Tc1 and Th1 cells. The induced PD-L1 is causatively associated with reduction of inflammation and intestinal leakage induced by anti-CTLA-4 antibodies as it is abrogated by anti-PD-1 antibody.

The ability of anti-PD-1 to abrogate protection by echinomycin also suggests an interesting explanation on how anti-PD-1 exacerbates irAE when used in conjunction with anti-CTLA-4: PD-L1 is induced by anti-CTLA-4-induced IFNγ as a negative feedback mechanism to control irAE. By preventing PD-L1 from interacting with PD-1, anti-PD-1/PD-L1 antibodies exacerbate irAE caused by anti-CTLA-4 antibodies. In contrast to anti-PD-1, echinomycin not only further enhanced anti-CTLA-4 induced PD-L1 in normal tissue, but also allows PD-L1 to signal through PD-1 to supercharge the immune tolerance checkpoint function.

While HIF-1α has been shown to be involved in degradation of Foxp3 and induce Th17 (Dang et al., 2011), its function in inducing Th1 has also been reported (Shehade et al., 2015). Our data presented herein show a strong effect of echinomycin in inducing IFNγ-producing cells, including Tc1 and Th1 cells. It is unclear whether echinomycin promotes Tc1 expansion in vivo by cell-intrinsic targeting of HIF-1α or indirectly by reduction of regulatory T cells. Regardless of whether the effect is T cell-intrinsic, the induction of PD-L1 normal tissues are tissue cell-extrinsic. In contrast, in cancer cells, targeting HIF-1α resulted in a cell-intrinsic inhibition of PD-L1. Thus, the data presented herein revealed a cancer cell-intrinsic inhibition of PD-L1 and normal tissue cell-extrinsic induction of PD-L1 by echinomycin. Together, these two activities provide what we believe is the first example in cancer immunotherapy of an approach which abrogates the PD-1-PD-L1 checkpoint in the TME to eliminate immune evasion by cancer cells, while fortifying its immune tolerance checkpoint activity in normal tissues. Therefore, HIF-1α inhibitors represents an effective immunotherapy, and an ideal partner for CTLA-4-targeted immunotherapy.

Materials & Methods

Cell Lines Murine tumor cell lines were obtained from American Type Culture Collection (Manassas, VA).

Therapeutic Agents Echinomycin was provided by Oncoimmune, Inc. (Rockville, MD) and formulated with liposomes as previously described (Bailey et al., 2020). Recombinant Ipilimumab with the amino acid sequenced disclosed in WC500109302 and http://www.drugbank.ca/drugs/DB06186 was provided by Lakepharma Inc. (San Francisco, CA). Anti-mouse CTLA-4 (clone 9D9), anti-mouse PD-1 (clone RMP1-14), and anti-mouse IFNγ (clone XMG1.2) were purchased from BioXCell (West Lebanon, NH).

Mice BALB/cAnNCr and C57BL/6NCr were obtained from NCI (Bethesda, MD), and NOD.Cg-Prkdc^{scid}Il2rγ^{tm1Wj1}/SzJ (NSG) mice were purchased from the University of Maryland Baltimore School of Medicine and bred in-house. Human CTLA4 knockin mice were produced and maintained in-house and have been previously described (Du et al., 2018). All procedures involving experimental animals were approved by the Institutional Animal Care and Use Committee (IACUC) of the University of Maryland School of Medicine.

Tumor Models The details of each experiment are specified in the figure legends. For 4T1 and E0771, 0.5-1.0×10⁶ cells suspended in RPMI-1640 medium were orthotopically injected into the first mammary fat pad on the left side of female recipient mice, aged 6-8 weeks, at 50 μl/mouse. For MC38, 1×10⁶ cells were injected subcutaneously into the left inguinal canal of male recipients, aged 6-8 weeks, at 50 μl/mouse. On day 6 after transplantation, mice with palpable tumors were assigned into different treatment groups in a manner to achieve comparable mean tumor volumes between experimental and control groups. Tumor volumes were calculated and reported based on the formula V=a²/2, where a is the longer diameter, and b is the shorter diameter. Echinomycin, or equivalent of empty liposomes as a vehicle control, were administered by intravenous (i.v.) injection into the lateral tail vein on the indicated days, at 0.15-0.25 mg/kg. Intraperitoneal (i.p.) injection was used to deliver therapeutic antibodies 9D9, RMP1-14, or XMG1.2 at 0.2 mg/mouse/injection. The mice from different groups were sacrificed at the same timepoints for analyses.

Statistics All experiments have been replicated at least twice, producing similar results. For each statistical analysis, appropriate tests were selected on the basis of whether the data with outlier deletion was normally distributed by using the D'Agostino & Pearson normality test. Unless otherwise noted in the figure legends, data were analyzed using an unpaired two-tailed Student's t test or a Mann-Whitney test to compare between two groups, one-way analysis of variance (ANOVA) with Sidak's posttest or Kruskal-Wallis test with Dunn's posttest for multiple comparisons, and two-way ANOVA for behavioral tests. The correlation coefficient and P-value of linear regression were calculated by Pearson's method. Sample sizes were chosen with adequate statistical power on the basis of the literature and past experience. In the graphs, data are shown as mean±SEM, indicated by horizontal line and y-axis error bars, respectively. Statistical calculations were performed using GraphPad Prism 8 software (GraphPad Software, San Diego, California). ns, not significant, *P<0.05, **P<0.01, ***P<0.001, ****P<0.0001.

### REFERENCES

Amaria, RN., Reddy, S. M., Tawbi, H. A., Davies, M. A., Ross, M. I., Glitza, I. C., Cormier, J. N., Lewis, C., Hwu, W. J., Hanna, E., et al. (2018). Neoadjuvant immune checkpoint blockade in high-risk resectable melanoma. Nat Med 24, 1649-1654.
Anderson, KG., Stromnes, I. M., and Greenberg, P. D. (2017). Obstacles Posed by the Tumor Microenvironment to T cell Activity: A Case for Synergistic Therapies. Cancer cell 31, 311-325.
Bailey, C. M., Liu, Y., Peng, G., Zhang, H., He, M., Sun, D., Zheng, P., Liu, Y., and Wang, Y. (2020). Liposomal formulation of HIF-1alpha inhibitor echinomycin eliminates established metastases of triple-negative breast cancer. Nanomedicine 29, 102278.
Barber, D. L., Wherry, E. J., Masopust, D., Zhu, B., Allison, J. P., Sharpe, A. H., Freeman, G. J., and Ahmed, R. (2006). Restoring function in exhausted CD8 T cells during chronic viral infection. Nature 439, 682-687.
Barsoum, LB., Smallwood, C. A., Siemens, D. R., and Graham, C. H. (2014). A mechanism of hypoxia-mediated escape from adaptive immunity in cancer cells. Cancer Res 74, 665-674.
Blank, C. U., Rozeman, E. A., Fanchi, L. F., Sikorska, K., van de Wiel, B., Kvistborg, P., Krijgsman, O., van den Braber, M., Philips, D., Broeks, A., et al. (2018). Neoadjuvant versus adjuvant ipilimumab plus nivolumab in macroscopic stage III melanoma. Nat Med 24, 1655-1661.
Dang, E. V., Barbi, J., Yang, H. Y., Jinasena, D., Yu, H., Zheng, Y., Bordman, Z., Fu, J., Kim, Y., Yen, H. R., et al. (2011). Control of T(H)17/T(reg) balance by hypoxia-inducible factor 1. Cell 146, 772-784.
Dong, H., Strome, S. E., Salomao, D. R., Tamura, H., Hirano, F., Flies, D. B., Roche, P. C., Lu, J., Zhu, G., Tamada, K., et al. (2002). Tumor-associated B7-H1 promotes T-cell apoptosis: a potential mechanism of immune evasion. Nat Med 8, 793-800.
Du, X., Liu, M., Su, J., Zhang, P., Tang, F., Ye, P., Devenport, M., Wang, X., Zhang, Y., Liu, Y., et al. (2018). Uncoupling therapeutic from immunotherapy-related adverse effects for safer and effective anti-CTLA-4 antibodies in CTLA4 humanized mice. Cell Res 28, 433-447.
Gordon, S. R., Maute, R. L., Dulken, B. W., Hutter, G., George, B. M., McCracken, M. N., Gupta, R., Tsai, J. M., Sinha, R., Corey, D., et al. (2017). PD-1 expression by tumour-associated macrophages inhibits phagocytosis and tumour immunity. Nature 545, 495-499.
Hellmann, M. D., Ciuleanu, T. E., Pluzanski, A., Lee, J. S., Otterson, G. A., Audigier-Valette, C., Minenza, E., Linardou, H., Burgers, S., Salman, P., et al. (2018). Nivolumab plus Ipilimumab in Lung Cancer with a High Tumor Mutational Burden. The New England journal of medicine 378, 2093-2104.
Hirano, F., Kaneko, K., Tamura, H., Dong, H., Wang, S., Ichikawa, M., Rietz, C., Flies, D. B., Lau, J. S., Zhu, G., et al. (2005). Blockade of B7-H1 and PD-1 by monoclonal antibodies potentiates cancer therapeutic immunity. Cancer Res 65, 1089-1096.
Hodi, F. S., Chesney, J., Pavlick, A. C., Robert, C., Grossmann, K. F., McDermott, D. F., Linette, G. P., Meyer, N., Giguere, J. K., Agarwala, S. S., et al. (2016). Combined nivolumab and ipilimumab versus ipilimumab alone in patients with advanced melanoma: 2-year overall survival outcomes in a multicentre, randomised, controlled, phase 2 trial. The Lancet Oncology 17, 1558-1568.
Iommarini, L., Porcelli, A. M., Gasparre, G., and Kurelac, I. (2017). Non-Canonical Mechanisms Regulating Hypoxia-Inducible Factor 1 Alpha in Cancer. Front Oncol 7, 286.
Iwai, Y., Ishida, M., Tanaka, Y., Okazaki, T., Honjo, T., and Minato, N. (2002). Involvement of PD-L1 on tumor cells in the escape from host immune system and tumor immunotherapy by PD-L1 blockade. Proc Natl Acad Sci USA 99, 12293-12297.
Iwai, Y., Terawaki, S., and Honjo, T. (2005). PD-1 blockade inhibits hematogenous spread of poorly immunogenic tumor cells by enhanced recruitment of effector T cells. Int Immunol 17, 133-144.
Larkin, J., Chiarion-Sileni, V., Gonzalez, R., Grob, J. J., Cowey, C. L., Lao, C. D., Schadendorf, D., Dummer, R., Smylie, M., Rutkowski, P., et al. (2015). Combined Nivolumab and Ipilimumab or Monotherapy in Untreated Melanoma. The New England journal of medicine 373, 23-34.
Larkin, J., Chiarion-Sileni, V., Gonzalez, R., Grob, J. J., Rutkowski, P., Lao, C. D., Cowey, C. L., Schadendorf, D., Wagstaff, J., Dummer, R., et al. (2019). Five-Year Survival with Combined Nivolumab and Ipilimumab in Advanced Melanoma. The New England journal of medicine 381, 1535-1546.
Luoma, A. M., Suo, S., Williams, H. L., Sharova, T., Sullivan, K., Manos, M., Bowling, P., Hodi, F. S., Rahma, O., Sullivan, R. J., et al. (2020). Molecular Pathways of Colon Inflammation Induced by Cancer Immunotherapy. Cell 182, 655-671 e622.
Mazanet, M. M., and Hughes, C. C. (2002). B7-H1 is expressed by human endothelial cells and suppresses T cell cytokine synthesis. J Immunol 169, 3581-3588.
Morganstein, D. L., Lai, Z., Spain, L., Diem, S., Levine, D., Mace, C., Gore, M., and Larkin, J. (2017). Thyroid abnormalities following the use of cytotoxic T-lymphocyte antigen-4 and programmed death receptor protein-1 inhibitors in the treatment of melanoma. Clinical endocrinology 86, 614-620.
Motzer, R. J., Tannir, N. M., McDermott, D. F., Aren Frontera, O., Melichar, B., Choueiri, T. K., Plimack, E. R., Barthelemy, P., Porta, C., George, S., et al. (2018). Nivolumab plus Ipilimumab versus Sunitinib in Advanced Renal-Cell Carcinoma. The New England journal of medicine 378, 1277-1290.
Muz, B., de la Puente, P., Azab, F., and Azab, A. K. (2015). The role of hypoxia in cancer progression, angiogenesis, metastasis, and resistance to therapy. Hypoxia (Auckl) 3, 83-92.
Naidoo, J., Wang, X., Woo, K. M., Iyriboz, T., Halpenny, D., Cunningham, J., Chaft, J. E., Segal, N. H., Callahan, M. K., Lesokhin, A. M., et al. (2017). Pneumonitis in Patients Treated With Anti-Programmed Death-1/Programmed Death Ligand 1 Therapy. J Clin Oncol 35, 709-717.
Nishimura, H., Nose, M., Hiai, H., Minato, N., and Honjo, T. (1999). Development of lupus-like autoimmune diseases by disruption of the PD-1 gene encoding an ITIM motif-carrying immunoreceptor. Immunity 11, 141-151.
Nishimura, H., Okazaki, T., Tanaka, Y., Nakatani, K., Hara, M., Matsumori, A., Sasayama, S., Mizoguchi, A., Hiai, H., Minato, N., et al. (2001). Autoimmune dilated cardiomyopathy in PD-1 receptor-deficient mice. Science 291, 319-322.
Noman, M. Z., Desantis, G., Janji, B., Hasmim, M., Karray, S., Dessen, P., Bronte, V., and Chouaib, S. (2014). PD-L1 is a novel direct target of HIF-1alpha, and its blockade under hypoxia enhanced MDSC-mediated T cell activation. J Exp Med 211, 781-790.
Peng, G., and Liu, Y. (2015). Hypoxia-inducible factors in cancer stem cells and inflammation. Trends in pharmacological sciences 36, 374-383.
Postow, M. A., Chesney, J., Pavlick, A. C., Robert, C., Grossmann, K., McDermott, D., Linette, G. P., Meyer, N., Giguere, J. K., Agarwala, S. S., et al. (2015). Nivolumab and ipilimumab versus ipilimumab in untreated melanoma. The New England journal of medicine 372, 2006-2017.
Sanmamed, M. F., and Chen, L. (2019). A Paradigm Shift in Cancer Immunotherapy: From Enhancement to Normalization. Cell 176, 677.
Semenza, G. L. (2003). Targeting HIF-1 for cancer therapy. Nat Rev Cancer 3, 721-732.
Shehade, H., Acolty, V., Moser, M., and Oldenhove, G. (2015). Cutting Edge: Hypoxia-Inducible Factor 1 Negatively Regulates Th1 Function. J Immunol 195, 1372-1376.
Strome, S. E., Dong, H., Tamura, H., Voss, S. G., Flies, D. B., Tamada, K., Salomao, D., Cheville, J., Hirano, F., Lin, W., et al. (2003). B7-H1 blockade augments adoptive T-cell immunotherapy for squamous cell carcinoma. Cancer Res 63, 6501-6505.
Talks, K. L., Turley, H., Gaffer, K. C., Maxwell, P. H., Pugh, C. W., Ratcliffe, P. J., and Harris, A. L. (2000). The expression and distribution of the hypoxia-inducible factors HIF-1alpha and HIF-2alpha in normal human tissues, cancers, and tumor-associated macrophages. Am J Pathol 157, 411-421.
Walunas, T. L., Lenschow, D. J., Bakker, C. Y., Linsley, P. S., Freeman, G. J., Green, J. M., Thompson, C. B., and Bluestone, J. A. (1994). CTLA-4 can function as a negative regulator of T cell activation. Immunity 1, 405-413.
Wang, C., Thudium, K. B., Han, M., Wang, X. T., Huang, H., Feingersh, D., Garcia, C., Wu, Y., Kuhne, M., Srinivasan, M., et al. (2014). In vitro characterization of the anti-PD-1 antibody nivolumab, BMS-936558, and in vivo toxicology in non-human primates. Cancer immunology research 2, 846-856.
Wang, D. Y., Salem, J. E., Cohen, J. V., Chandra, S., Menzer, C., Ye, F., Zhao, S., Das, S., Beckermann, K. E., Ha, L., et al. (2018). Fatal Toxic Effects Associated With Immune Checkpoint Inhibitors: A Systematic Review and Meta-analysis. JAMA oncology 4, 1721-1728.
Wang, Y., Liu, Y., Malek, S. N., Zheng, P., and Liu, Y. (2011). Targeting HIF1alpha eliminates cancer stem cells in hematological malignancies. Cell stem cell 8, 399-411.
Waterhouse, P., Penninger, J. M., Timms, E., Wakeham, A., Shahinian, A., Lee, K. P., Thompson, C. B., Griesser, H., and Mak, T. W. (1995). Lymphoproliferative disorders with early lethality in mice deficient in Ctla-4 [see comments]. Science 270, 985-988.
Yang, H. R., Chou, H. S., Gu, X., Wang, L., Brown, K. E., Fung, J. J., Lu, L., and Qian, S. (2009). Mechanistic insights into immunomodulation by hepatic stellate cells in mice: a critical role of interferon-gamma signaling. Hepatology 50, 1981-1991.
Zerdes, I., Matikas, A., Bergh, J., Rassidakis, G. Z., and Foukakis, T. (2018). Genetic, transcriptional and post-translational regulation of the programmed death protein ligand 1 in cancer: biology and clinical correlations. Oncogene 37, 4639-4661.

1. A method of treating a cancer in a subject in need of cancer immunotherapy, comprising administering a HIF-1α inhibitor to the subject.

2. The method of claim 1, wherein the HIF-1α inhibitor is echinomycin.

3. The method of claim 1 or 2, wherein the cancer is PD-L1-positive.

4. The method of claim 3, wherein the cancer is selected from the group consisting of: melanoma, lung cancer, non-small cell lung cancer, small cell lung cancer, squamous cell lung carcinoma, Hodgkin's lymphoma, classical Hodgkin's lymphoma, hairy leukemia, colorectal cancer, liver cancer, urothelial carcinoma, bladder cancer, renal cancer, renal cell carcinoma, kidney cancer, prostate cancer, head and neck squamous cell carcinoma, breast cancer, Merkel cell carcinoma, hepatocellular carcinoma, gastric cancer, advanced solid or hematologic malignancy, chronic lymphocytic leukemia, multiple myeloma, acute myeloid leukemia, MSI-high cancer, cervical cancer, mediastinal B-cell lymphoma, ovarian cancer, triple negative breast cancer, pancreatic cancer, glioblastoma, and medulloblastoma.

5. The method of any one of claims 1-4, wherein the method targets Tregs in the tumor microenvironment (TME).

6. The method of claim 5, wherein the method abrogates PD-L1 in the TME, and induces PD-L1 in normal tissues.

7. The method of any one of claims 1-6, wherein the HIF-1α inhibitor is administered at a dose of about 100 to 1000 μg/m² as measured by body surface area (BSA).

8. A method of treating a cancer in a subject in need of cancer immunotherapy, comprising administering a HIF-1α inhibitor and a second cancer immunotherapeutic agent to the subject.

9. The method of claim 8, wherein the HIF-1α inhibitor is echinomycin.

10. The method of claim 8 or 9, wherein the second cancer immunotherapeutic agent is an anti-CTLA-4 antibody.

11. The method of claim 10, wherein the anti-CTLA-4 antibody is Ipilimumab or Tremelimumab.

12. The method of any one of claims 8-11, wherein the cancer is PD-L1-positive.

13. The method of any one of claims 8-12, wherein the cancer is characterized by significant infiltration of regulatory T-cells.

14. The method of claim 12 or 13, wherein the cancer selected from the group consisting of: melanoma, lung cancer, non-small cell lung cancer, small cell lung cancer, squamous cell lung carcinoma, Hodgkin's lymphoma, classical Hodgkin's lymphoma, hairy leukemia, colorectal cancer, liver cancer, urothelial carcinoma, bladder cancer, renal cancer, renal cell carcinoma, kidney cancer, prostate cancer, head and neck squamous cell carcinoma, breast cancer, Merkel cell carcinoma, hepatocellular carcinoma, gastric cancer, advanced solid or hematologic malignancy, chronic lymphocytic leukemia, multiple myeloma, acute myeloid leukemia, MSI-high cancer, cervical cancer, mediastinal B-cell lymphoma, ovarian cancer, triple negative breast cancer, pancreatic cancer, glioblastoma, and medulloblastoma.

15. The method of claim 14, wherein the cancer is a melanoma, non-small cell lung carcinoma, small cell lung cancer, squamous cell lung carcinoma, bladder cancer, renal cancer, breast cancer, liver cancer, pancreatic cancer, ovarian cancer, colorectal cancer, gastric cancer, or prostate cancer.

16. The method of any one of claims 8-15, wherein the HIF inhibitor is administered at a dose of about 100 to 1000 μg/m² as measured by body surface area (BSA).

17. The method of any one of claims 8-16, wherein the immunotherapy targets Tregs in the tumor microenvironment (TME).

18. The method of claim 17, wherein the method abrogates PD-L1 in the TME, and induces PD-L1 in normal tissues.

19. The method of any one of claims 10-18, wherein the treatment comprising the HIF-1α inhibitor and the anti-CTLA-4 antibody exhibits improved safety as compared to combination cancer immunotherapy comprising an anti-PD-L1 antibody and the anti-CTLA-4 antibody.

20. The method of claim 19, wherein the improved safety is fewer immune related adverse events, as measured in a population of subjects treated with the combination of the HIF-1α inhibitor and the anti-CTLA-4 antibody as compared to a population of subjects treated with the anti-PD-L1 antibody and the anti-CTLA-4 antibody.

21. The method of claim 20, wherein the anti-CTLA-4 antibody is Ipilimumab and the HIF-1α inhibitor is echinomycin.
